(19) United States
(12) Patent Application Publication
(10) Pub. No.: US 2022/0349906 A1
(43) Pub. Date: Nov. 3, 2022

(54) FILTRATION-BASED METHODS FOR PREPARING FETAL NUCLEATED RED BLOOD CELLS (NRBCS) FOR DIAGNOSTIC TESTING

(71) Applicant: KELLBENX INCORPORATED, Great River, NY (US)

(72) Inventor: Hassan Bennani, Dix Hills, NY (US)

(73) Assignee: KELLBENX INCORPORATED, Great River, NY (US)

(21) Appl. No.: 17/607,978

(22) PCT Filed: May 1, 2020
(86) PCT No.: PCT/US2020/030947

§ 371 Date: Nov. 1, 2021

(51) Int. Cl. G01N 33/80; C12Q 1/6841; G01N 1/34; G01N 1/30; G01N 21/64

(52) U.S. Cl. CPC G01N 33/80; C12Q 1/6841; G01N 1/34; G01N 1/30; G01N 21/6458; G01N 21/6428; G01N 2800/38; G01N 2021/6439

(57) ABSTRACT

The disclosure relates to methods of preparation of fetal nucleated red blood cells (NRBCs) from biological samples for diagnostic testing.

### 1. CROSS-REFERENCE TO RELATED APPLICATIONS

This application claims the priority benefit of U.S. provisional application No. 62/842,094, filed May 2, 2019 the contents of which are incorporated herein in their entireties by reference thereto.

### 2. BACKGROUND

The practice of prenatal diagnosis to detect possible chromosomal and genetic abnormalities of the fetus enables parents and caregivers to initiate monitoring of predispositions and early treatment of diseases or conditions. The practice of prenatal diagnosis has been established to detect possible chromosomal and genetic abnormalities of the fetus, thus enabling informed decisions by the parents and the care givers. Among various chromosomal abnormalities compatible with life (aneuploidy 21, 18, 13, X, Y), Down syndrome, caused by the presence of all or part of an extra copy chromosome 21, is the most common genetic cause of mental retardation and the primary reason for women seeking prenatal diagnosis (Pierce B. Genetics: A conceptual approach (W. H. Freeman and company, 2008), 3d edition; Driscoll and Gross, 2009, N Engl J Med. 360:2556-62). Cytogenetic disorders reportedly occur in about 1% of live births, 2% of pregnancies in women older than 35 years, and in approximately 50% of spontaneous first trimester miscarriage (Thompson and Thompson Genetics in Medicine, sixth edition, chapter 9). The incidence of single gene defects in a population of one million live births is reportedly about 0.36% (Thompson and Thompson Genetics in Medicine, sixth edition, chapter 9).

The preferred first trimester screening, involving quantification from serum of PAPP-A (pregnancy-associated plasma-protein-A), free β-Hcg (free β-human chorionic gonadotrophins), and ultrasound examination of nuchal translucency, has a Down syndrome detection rate of about 90%, but at the expense of a significant 5% false positive rate (Nicolaides et al., 2005, Ultrasound Obstet Gynecol 25:221-26). A meta-analysis of first trimester screening studies (Evans et al., 2007, Am J Obstet Gynecol 196:198-05) concluded that in practice the achievable sensitivity might be significantly lower (about 80-84%) than reported.

Definitive detection of chromosomal abnormalities and single gene disorders is possible by karyotype analysis of fetal tissues obtained by chorionic villus sampling, amniocentesis or umbilical cord sampling. To minimize risks of conditions such as Down syndrome, these tests are offered to women identified by a set of screening criteria as having the highest risk for fetal chromosomal abnormalities. This group generally includes pregnancies with mal [symbol] or older and abnormal responses to ultrasound examinations of the fetus and/or [symbol] serum marker screening tests performed during first and/or second trimesters of pregnancy (Nicolaides et al., 2005, Ultrasound Obstet Gynecol 25:221-26). However, these procedures are highly invasive, require skilled professionals, and are prone to significant risk of fetal loss (up to 1%) and/or maternal complications (Mujezinovic et al., 2007, Obstet Gynecol 110:687-94; Tabor et al., 1986, Lancet 1:1287-93; Buscaglia et al., 1996, Prenat Diagn 16:375-76). A guideline by the American College of Obstetricians and Gynecologists (ACOG) advising its members to test all expected mothers for genetic abnormalities (ACOG Practice bulletin Clinical Management Guidelines for Ob-Gyns, No. 7, Jan 2007) is an indication of the unmet need for non-invasive technologies that could safely lead to specific diagnosis of fetal genetic status.

For several decades, the search for non-invasive alternatives has focused on isolation, identification, and subsequent analysis of fetal genetic materials that normally cross the placental barrier into maternal circulation. Since the pioneering reports on detection of fetal cells in 1893 and later of fetal cell-free DNA and in maternal blood (see Table 1 of Purwosunu et al., 2006, Taiwanese J. Obstet Gynecol 45(1):10-20, two promising approaches based on analysis of fetal cells or cell free fetal genetic materials has received tremendous interest.

“Cell-free” fetal DNA is relatively abundant in maternal blood, constituting 5-10% of the total cell-free DNA in maternal plasma (Hahn et al., 2011, Expert Reviews in Molecular Medicine 13:e16). Cell-free DNA-based prenatal testing, which become viable with the advent of next generation sequencing techniques, first became commercially available in the U.S. in 2011, and at least four such assays are currently commercialized. To date cell-free DNA testing methods permit gender identification, aneuploidy detection and mutations present in paternal DNA, but not more refined genetic analyses, such as detection of microdeletions or microinsertions (see, e.g., Simpson, 2013, Fertility and Sterility 99:1124-1134). Moreover, inaccurate test results, including false positives, though infrequent, have been reported (see Simpson, 2013, Fertility and Sterility 99(4):1124-1134; Dugo et al., 2014, J Prenat Med. 8(1-2):31-35).

In comparison to cell-free fetal DNA or RNA, intact fetal cells can provide access to complete fetal genetic materials important for detection of chromosomal abnormalities as well as a more complete assessment of fetal genetic status (Huang et al., 2011, J Cell Biochem. 112:1475-85). A number of significant challenges have hampered development of reliable fetal cell isolation methods. The major limitation for isolation is the low number of circulating fetal nucleated cells in maternal blood, with estimates ranging from 1-2 fetal cells per mL of maternal blood (Bianchi et al., 1997, Am J Hum Genet 61(4):822-829) to 2-6 per mL of maternal blood (Krabchi et al., 2001, Clin Genet 60:145-150), although the numbers have been reported to be up to six-fold greater in aneuploidy pregnancies (Krabchi et al., 2006, Clin Genet 69:145-154 and Bianchi et al., 1997, Am J Hum Genet 61(4):822-829). To put this number i [symbol] the ratio of fetal cells to maternal cells in blood has been estimated at 1 in 10⁵ to [symbol] (See Purwosunu et al., 2006, Taiwanese J. Obstet Gynecol 45(1):10-20; Simpson, 2013, Fertility and Sterility 99(4):1124-1134), and for each 1-6 fetal cells in 1 mL of maternal blood there are approximately 4.2-5.4×10⁹ adult red blood cells, 1.16-8.3×10³ neutrophils, 2-9.5×10⁵ monocytes, 1-b 4.8×10⁶ lymphocytes, 1.33-3.33×10⁸ platelets, up to 4.5×10⁵ eosinophils and up to 2×10⁵ basophils (numbers taken from Uthman, Blood Cells and the CBC, which can be accessed at web2.iadfw.net/uthman/blood_cells.html).

Among variety of fetal cells in maternal blood (trophoblasts, lymphocytes, nucleated red blood cells, and hematopoietic stem cells; see Bianchi, 1999, Br J Haematol 105:574-83), nucleated red blood cells (NRBCs), known also as erythroblasts, have most of the desired characteristics for a reliable prenatal assay. Fetal NRBCs (fNRBCs) have limited life span and proliferative capacity (and therefore do not persist from one pregnancy to another), are mononucleated, carry a representative complement of fetal chromosomes, and are consistently present in maternal blood (Huang et al., 2011, J Cell Biochem. 112:1475-85; Kavanagh et al., 2010, J Chromat B 878:1905-11; Bianchi, 1999, Br J Haematol 105:574-83; Choolani et al., 2003, Mol Hum Repro 9:227-35; Bianchi and Lo, 2010, in Genetic Disorders and the Fetus: Diagnosis, Prevention and Treatment, Sixth Edition, Ch. 30, pp. 978-1000 (Milunsky and Milunsky eds.)). Studies of fetal erythropoiesis have identified two distinct processes, occurring initially in yolk sack (primitive erythropoiesis, producing primitive erythroblasts) and subsequently in fetal liver and bone marrow (producing definitive erythroblasts) (Huang et al., 2011, J Cell Biochem. 112:1475-85). Both primitive and definitive erythroblasts have been detected in maternal circulation, with primitive erythroblasts being the predominant first trimester cell type that is progressively replaced by the definitive type that persists until term (Huang et al., 2011, J Cell Biochem. 112:1475-85; Choolani et al., 2003, Mol Hum Repro 9:227-35).

The most extensive study of fetal cells in maternal blood was the multi-year, multi-center NIFTY Trial, which was designed to evaluate the utility and feasibility of isolating fetal cells to diagnose fetal abnormalities. The four centers involved attempted to isolate fetal cells from maternal blood and analyze the isolated cells by fluorescent in situ hybridization (FISH) with chromosome-specific probes (Bianchi et al., 2002, Prenat Diagn 022:609-615). The four centers, designated A, B, C and D, all used density gradient separation as a preliminary step to deplete maternal cells and then used different methods to obtain fetal cells for FISH. At center A, density separation was followed by cell fixation, negative selection by MACS using anti-CD14 and anti-CD15 antibodies, and FACS using anti-HbF (fetal hemoglobin). At center B, density gradient separation was followed by cell fixation and simultaneous negative and positive selection using FACS with anti-HbF antibodies for positive selection and anti-CD45 or anti-HbA (adult hemoglobin) for negative selection. At center C, density gradient separati [symbol] by negative selection by MACS using anti-CD14 and anti-CD45 antibodies, FACS using anti-CD71 antibodies, and cell fixation. At center D, density gradient separation was followed by cell fixation and positive selection using MACS with anti-CD71 antibodies. The general detection rate of X and Y chromosomes in male fetal cells was only 41.1% of cases, and the false positive rate (i.e., detection of X and Y chromosomes in female fetal cells) was 11.1%. The overall detection rate of aneuploidies was 74.4%, with a false positive rate estimated to be between 0.6% and 4.1%. See Bianchi et al., 2002, Prenat Diagn 22:609-615. The MACS-based methods were said to provide better recovery and detection than FACS-based methods (Bianchi and Lo, 2010, in Genetic Disorders and the Fetus: Diagnosis, Prevention and Treatment, Sixth Edition, Ch. 30, pp. 978-1000 (Milunsky and Milunsky eds.)). One of the NIFTY Trial's contributors stated that the approach “was laborious, lacked consistent recovery, and had an unacceptable non-informative rate.” Simpson, 2013, Fertility and Sterility 99(4):1124-1134.

A variety of other approaches have to been utilized to isolate fetal cells, including centrifugation, filtration, lateral displacement, magnetophoresis, lectin-binding, dielectrophoresis, micromanipulation and laser capture, and microdissection. Higher throughput methods, such as microelectronic mechanical systems (MEMS) and automated cell enrichment methods, have also been utilized (Kavanagh et al., 2010. J Chromat B 878:1905-11; Kilpatrick et al., 2004, J Obstet Gynecol 190:1571-81; Seppo et al., 2008, Prenat Diagn 28:815-21; Talasaz et al., 2009, PANS 106:3970-75, 2009; Kumo et al., 2010, 14th International Conference on Miniaturized Systems for Chemistry and Life Sciences. 3-7 October 2010, Groningen, The Netherlands; pp. 1583-1585; Cheng et al., 2011, J Clin Lab Anal 25:1-7; Choolani et al., 2012, Best Practice & Research Clinical Obstetrics and Gynaecology 26:655-667). These too have provided inconsistent results (Simpson, 2013, Fertility and Sterility 99(4):1124-1134).

There is a need for improved fetal cell isolation techniques that permit downstream genetic analysis of fetal DNA.

### 3. SUMMARY

The present disclosure is based on the development of isolation techniques that permit enrichment and isolation of fetal nucleated red blood cells (fNRBCs) from a mixed cell population in which the fNRBCs are a small minority. Accordingly, the present disclosure provides cell preparations highly enriched for fNRBCs and methods of producing such enriched cell populations.

The present disclosure is based, in part, on the use of filtration to enrich for fNRBCs from a biological sample, such as maternal blood or an fNRBC-enriched cell fraction of maternal blood. Filters that can be used in the methods of the disclosure include [symbol] leukocyte reduction filters such as the Pall Purecell® NEO filter. The maternal blood is typically drawn in the time period starting at around four weeks of gestation.

The enrichment methods of the disclosure can be used in conjunction with one or more positive selection methods that deplete other cell types, e.g., maternal lymphocytes or red blood cells, from the biological sample. Positive selection methods that can be used in the methods of the disclosure include magnetic activated cell sorting (MACS). The use of a positive selection method following filtration can further reduce the number of maternal cells, e.g., maternal leukocytes, remaining in a sample following filtration.

Once a preparation of cells enriched in fNRBCs is made, the preparation itself can be subject to diagnostic testing, or additional isolation techniques (e.g., micromanipulation) can be utilized to select individual fNRBCs or groups of fNRBCs for diagnostic testing. One or more of the fNRBCs can be subject to a validation technique, such as short tandem repeat (“STR”) analysis, to confirm the identity of a cell as a fetal cell.

In some aspects, the present disclosure provides a method for preparing fNRBCs, comprising subjecting a biological sample comprising fNRBCs to filtration, and then selecting individual fNRBCs or groups of fNRBCs by micromanipulation. The methods in some embodiments include a positive selection step following the filtration. The positive selection preferably includes positive immunoselection and optionally one or more additional positive selection criteria. The positive immunoselection typically comprises the steps of: (a) contacting the biological sample with one or more positive immunoselective antibodies (e.g., one, two, three or more positive immunoselective antibodies) in a fluid medium, wherein the positive immunoselective antibody selectively binds to fNRBCs relative to one or more other cell types in the biological sample; and (b) selecting cells bound to said positive immunoselective antibody/antibodies. Illustrative embodiments of positive selection into which the foregoing positive selection steps can be incorporated are described in Sections 5.3 and 6.2.

In certain aspects, at least one positive immunoselective antibody binds an antigen present on the surface of fNRBCs but does not bind CD71 or other surface antigens present on adult erythroid cells. In some embodiments, the positive immunoselective antibody is 4B9 or an antibody that competes with 4B9 for binding to the surface of fNRBCs. Other markers for positive selection can include glycophorin A (also known as CD235a), CD36, CD71, and nuclear stains (e.g., Hoechst 33342, LDS751, TO-PRO, DC-Ruby, Cy5 and DAPI). An immunoselection step can utilize magnetic separation, e.g., using antibody-coated magnetic beads, or flow cytometry. Flow cytometric techniques can provide accurate separation via the use of, e.g., fluorescence activated cell sorters, which can have varying degrees of sophistication, such as multiple color channels, low angle and obtuse light scattering detecting channels, impedance channels, etc. Accordingly, as used herein, the term “flow cytometry” encompasses fluorescent activated cell sorting (FACS). Preferably, the methods [symbol] disclosure comprise MACS.

Once a preparation of cells enriched in fNRBCs is made, the preparation itself can be subject to a diagnostic assay, or additional isolation techniques (e.g., micromanipulation, capture of the cells on a solid surface) can be utilized to select individual fNRBCs or pools of fNRBCs for diagnostic testing. In some embodiments, the additional isolation techniques (e.g., micromanipulation) can take advantage of the fluorescent label(s) utilized to enrich the cells and/or additional fluorescent labels added to the cells following enrichment, the presence of hemoglobin in the fNRBCs (detectable by a Soret band filter) and fNRBC morphological features (Huang et al., 2011, J Cell Biochem. 112:1475-85; Choolani et al., 2003, Mol Hum Repro 9:227-35). Exemplary approaches for micromanipulation are described in Section 5.4.

The present disclosure further provides preparations of fNRBCs prepared or obtainable by the methods described herein, including individual fNRBCs or groups of fNRBCs isolated by the methods described herein. Exemplary fNRBC populations are described in Section 5.5.

The fNRBCs can be used in fetal diagnostic testing, e.g., for determining the presence of a fetal abnormality. Examples of abnormalities that can be tested for include trisomy 13, trisomy 18, trisomy 21, Down syndrome, neuropathy with liability to pressure palsies, neurofibromatosis, Alagille syndrome, achondroplasia, Huntington's disease, alpha-mannosidosis, beta-mannosidosis, metachromatic leucodystrophy, von Recklinghausen's disease, tuberous sclerosis complex, myotonic dystrophy, cystic fibrosis, sickle cell disease, Tay-Sachs disease, beta-thalassemia, mucopolysaccharidoses, phenylketonuria, citrullinuria, galactosemia, galactokinase and galactose 4-epimerase deficiency, adenine phosphoribosyl, transferase deficiency, methylmalonic acidurias, proprionic acidemia, Farber's disease, fucosidosis, gangliosidoses, gaucher's disease, I cell disease, mucolipidosis III, Niemann-Pick disease, sialidosis, Wolman's disease, Zellweger syndrome, cystinosis, factor X deficiency, ataxia telangiectasia, Bloom's syndrome, Robert's syndrome, xeroderma pigmentosum, fragile (X) syndrome, sex chromosome aneuploidy, Klinefelter's Syndrome, Turner's syndrome, XXX syndrome, steroid sulfatase deficiency, microphthalmia with linear skin defects, Pelizaeus-Merzbacher disease, testis-determining factor on Y, ornithine carbamoyl transferase deficiency, glucose 6-phosphate dehydrogenase deficiency, Lesch-Nyhan syndrome, Anderson-Fabry disease, hemophilia A, hemophilia B, Duchenne type muscular dystrophy, Becker type muscular dystrophy, dup(17)(p11.2p11.2) syndrome, 16p11.2 deletion, 16p11.2 duplication, Mitochondrial defect, dup(22)(q11.2q11.2) syndrome, Cat eye syndrome, Cri-du-chat syndrome, Wolf-Hirschhorn syndrome, Williams-Beuren syndrome, Charcot-Marie-Tooth disease, chromosome rearrangements, chromosome deletions, Smith-Magenis syndrome, Velocardiofacial syndrome, DiGeorge syndrome, 1p36 deletion, Prader-Willi syndrome, Azospermia (factor a), Azospermia (factor b), Azospermia (factor c), spina bifida, anencephaly, neural tube defect, microcephaly, hydrocephaly, renal agenesis, Kallmann syndr [symbol] hypoplasia, Angelman syndrome, cystic kidney, cystic hygroma, fetal hydrops, ex [symbol] and gastroschisis, diaphragmatic hernia, duodenal atresia, skeletal dysplasia, cleft lip, cleft palate, argininosuccinicaciduria, Krabbe's disease, homocystinuria, maple syrup urine disease, 3-methylcrotonyl coenzyme A, carboxylase deficiency, Glycogenoses, adrenal hyperplasia, hypophosphatasia, placental steroid sulphatase deficiency, severe combined immunodeficiency syndrome, T-cell immunodeficiency, Ehlers-Danlos syndrome, osteogenesis imperfect, adult polycystic kidney disease, Fanconi's anemia, epidermolysis bullosa syndromes, hypohidrotic ectodermal dysplasia, congenital nephrosis (Finnish type) and multiple endocrine neoplasia.

The diagnostic assay can be a nucleic acid (e.g., DNA or RNA) assay, a protein (e.g., antibody-based) assay, or a histology assay, or a combination thereof. Examples of DNA assays include FISH, PCR and DNA sequencing assays. Examples of RNA assays include RT-PCR assay and FISH assays. To facilitate access to the nucleic acid, the fNRBCs can be lysed or permeabilized prior to carrying out the diagnostic test. The DNA, RNA and protein assays can be performed on a microarray. Exemplary techniques for molecular diagnostic testing are described in Section 5.7.

The diagnostic assay can be preceded, accompanied or followed by a molecular validation technique to confirm the identity of the cell or cell population being diagnosed as fetal cell(s). Exemplary validation techniques are described in Section 5.6.

The methods described herein can be performed once or multiple times during a given pregnancy, e.g., to confirm a particular diagnosis or to detect changes in the pregnancy or the condition of the fetus.

Kits useful for practicing the methods of the disclosure are described in Section 5.8.

### 4. BRIEF DESCRIPTION OF THE FIGURES

FIG. 1: FIG. 1 shows a heat map generated from fluorescence imaging of a petri dish containing a population of cells enriched for fNRBCs according to an exemplary method of the disclosure and stained with antibody 4B9, an anti-CD235a antibody, and a nuclear stain. The dots in FIG. 1 indicate areas of the petri dish containing “triple positive” cells stained with antibody 4B9, the anti-CD235a antibody, and the nuclear stain.

FIGS. 2A-2F: FIGS. 2A-2F show a nucleated red blood cell isolated according to an exemplary method of the disclosure viewed with various filters. FIG. 2A shows Alexa 488 staining; FIG. 2B shows staining for phycoerythrin (PE); FIG. 2C shows staining for Cy5; FIG. 2D shows the cell viewed in the Soret band; FIG. 2E shows the cell viewed under bright field illumination; FIG. 2F shows a combined image of FIGS. 2A-2E.

FIGS. 3A-3F: FIGS. 3A-3F show a nucleated red blood cell isolated acc [symbol] exemplary method of the disclosure viewed with various filters. FIG. 3A shows Alex 488 staining; FIG. 3B shows staining for phycoerythrin (PE); FIG. 3C shows staining for DAPI; FIG. 3D shows the cell viewed in the Soret band; FIG. 3E shows the cell viewed under bright field illumination; FIG. 3F shows a combined image of FIGS. 3A-3E.

### 5. DETAILED DESCRIPTION

5.1. Definitions

An antibody is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also any antigen binding fragment thereof (i.e., “antigen-binding portion”) or single chain thereof, fusion proteins comprising an antibody, and any other modified configuration of the immunoglobulin molecule that comprises an antigen recognition site, including, for example without limitation, single chain (scFv) and domain antibodies (e.g., human, camelid, or shark domain antibodies), maxibodies, minibodies, intrabodies, diabodies, triabodies, tetrabodies, vNAR and bis-scFv (see e.g., Hollinger and Hudson, 2005, Nature Biotech 23:1126-1136). An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. “Antibody” also encompasses any of each of the foregoing antibody/immunoglobulin types that has been modified to facilitate sorting and detection, for example as described in Section 5.3.4.

Antigen binding portion of an antibody, as used herein, refers to one or more fragments of an intact antibody that retain the ability to specifically bind to a given antigen (e.g., target X). Antigen binding functions of an antibody can be performed by fragments of an intact antibody. Examples of binding fragments encompassed within the term “antigen binding portion”

Biological sample is a sample in which fNRBCs are present or suspected to be present. In a particular embodiment, the biological sample is maternal blood or a fraction thereof enriched for fNRBCs (e.g., a fraction from which maternal non-nucleated red blood cells have been depleted). The maternal blood is typically drawn at 4 weeks, 5 weeks, 6 weeks, 8 weeks, 10 weeks, 12 weeks, 16 weeks, 20 weeks, 24 weeks, 30 weeks or 38 weeks of gestation, or one or more times during a time period ranging between any two of the foregoing embodiments, e.g., 4-38 weeks, 4-10 weeks, 4-16 weeks, 4-24 weeks, 5-16 weeks, 5-24 weeks, 5-38 weeks, 6-12 weeks, 6-16 weeks, 6-30 weeks, 6-20 weeks, 8-38 we [symbol] and so forth. The optimal period of gestation for drawing maternal blood for fNRBC enrichment is about 6 weeks to about 20 weeks of gestation. During this period, both primitive and definitive fetal red blood cells are present in the maternal circulation, thereby maximizing the quantities of fNRBCs enriched by the methods of the disclosure. The maternal blood can be from a single or multiple pregnancy (e.g., twins, triplets, quadruplets) and can include fNRBCs of a single gender (male or female) or both genders. Other types of biological samples are plasma, cells from a chorionic villus sampling (CVS) biopsy or cells from a percutaneous umbilical cord blood sampling, or a fraction thereof. As used herein, a “biological sample” can include reagents used in the enrichment or isolation of fNRBCs, such as buffers, antibodies and nuclear stains.

Chase refers to a step performed following filtration of the majority of a biological sample through a filter whereby a buffer (e.g., a PBS buffer) is mixed with the remainder of the biological sample that has not yet passed through the filter, and the mixture is then passed through the filter in the same direction as the previously filtered biological sample. For example, when filtering a biological sample contained in a blood bag through a leukocyte reduction filter, the sample can be passed from the blood bag through the filter until a small amount of the biological sample remains in the blood bag. Then, a chase can be performed by adding an amount of a buffer to the blood bag, mixing the buffer with the biological sample in the bag, and passing the mixture through the filter. Multiple chases (e.g., two or three) can be performed to increase the yield of a filtration step.

Compete, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present disclosure.

Negative selection refers to depletion of cells other than a target cell of interest from mixed cell population. Negative selection can be based on a marker that is absent from (or undetectable in or on) the target cell. Negative selection can also be based on o [symbol] e.g., size, morphology, or other physical characteristics.

Negative immunoselection refers to depletion of cells utilizing an antibody, e.g., an antibody that selectively binds to one or more cell types other than the target cells of interest but does not specifically bind to the target cells.

A negative immunoselective antibody is an antibody that can be used in negative immunoselection, e.g., is an antibody that binds to a marker that is present on or in one or more cell types other than the target cells but is absent from the target cell. The antibody can bind to a marker on the cell surface or an internal marker, but the marker is preferably a surface marker to avoid the need for fixation.

Positive selection refers to selection of cells (e.g., for enrichment and/or isolation purposes) containing a target cell of interest from a mixed cell population. Positive selection can be based on a marker that is present on or in the target cell. In some embodiments, the marker absent from (or undetectable in or on) one or more cell types (other than the target cell) in the population (e.g., biological sample) from which the target cell is to be isolated or enriched (for example, maternal blood or a fraction of maternal blood when the target cell is an fNRBC). In further embodiments, the marker absent from (or undetectable in or on) any cell type other than the target cell of interest in the population from which the target cell is to be isolated or enriched. Positive selection can also be based on other criteria, e.g., size, morphology, or other physical characteristics.

Positive immunoselection refers to selection of cells utilizing an antibody, e.g., an antibody that binds to a marker that is present on or in the target cell of interest and which is therefore useful for positive selection.

A positive immunoselective antibody is an antibody that can be used in positive immunoselection, e.g., is an antibody that binds to a marker that is present on or in the target cell. In some embodiments, the antibody selectively binds to the target cell but does not specifically bind to one or more other cell types that may be present in a population of cells in which the target cell is present. The antibody can bind to a marker on the cell surface or an internal marker, but the marker is preferably a surface marker to avoid the need for fixation.

Selective binding with respect to a particular cell refers to the specific or preferential binding of an antibody to a marker present in or on at least one cell type in a mixed cell population (e.g., a biological sample) but absent from (or undetectable in or on) at least one other cell type in the population. By way of example, if in a mixed cell population containing cell types A, B, C, D, and E, an antibody only specifically binds to cell type A or cell types A and E, the antibody is said to selectively bind to cell types A or cell types A and E, respectively.

An antibody specifically binds or preferentially binds to a target if it bi [symbol] affinity, avidity, more readily, and/or with greater duration than it binds to other substances. For example, an antibody that specifically or preferentially binds to a marker present on fNRBCs is an antibody that binds this marker with greater affinity, avidity, more readily, and/or with greater duration than it binds to other markers. Specific binding or preferential binding does not necessarily require (although it can include) exclusive binding. Generally, but not necessarily, reference to “binding” means preferential binding.

5.2. Pre-Enrichment

A pre-enrichment step prior to the filtration step of the enrichment and isolation methods of the disclosure is not required and in some embodiments the methods of the disclosure are performed in the absence of a pre-enrichment step. However, a pre-enrichment step can be performed if desired. Exemplary pre-enrichment processes are described below.

Density separation is a technique that allows the separation of cells depending on their size, shape and density. A density gradient is created in a centrifuge tube by layering solutions of varying densities with the dense end at the bottom of the tube. Cells are usually separated on a shallow gradient of sucrose or other inert carbohydrates even at relatively low centrifugation speeds.

Discontinuous density gradient centrifugation is commonly used to isolate peripheral blood mononuclear cells from granulocytes and erythrocytes. For example in a so called Ficoll density separation whole blood is layered over FICOLL-PAQUE® and then centrifuged. The erythrocytes, granulocytes and a portion of the mononuclear cells settle to the cell pellet while the remaining mononuclear cells settle to the Ficoll plasma interface.

Alternatively, adult red blood cells can be aggregated for depletion from a biological sample, permitting enrichment of a mononuclear cell fraction containing fNRBCs. If anti-coagulated blood is allowed to settle in a tube, erythrocytes sediment ahead of white blood cells, and a leukocyte-rich plasma layer may be removed after 1.5 hours or more. The erythrocytes sediment more rapidly than leukocytes because of the spontaneous tendency of erythrocytes to agglomerate. It is possible to accelerate the sedimentation of erythrocytes by adding an aggregation reagent. Exemplary aggregation reagents are nonionic polymers such as polysaccharides and synthetic polymers. In some embodiments, the polymers are dextrans of molecular weights 60,000-500,000, polyvinylpyrrolidone of molecular weigh 360,000, and polyoxyethylene (POE) of molecular weight 20,000. The aggregation reagents can be added to a biological sample containing buffer.

5.3. fNRBC Enrichment

The methods of the disclosure entail filtering a biological sample to enrich fNRBCs. The methods can also comprise one or more positive selection processes for enrichment and/or isolation of fNRBCs and typically entail at least one positive immunoselection step using antibodies that bind to fNRBCs. The filtration steps and positive selection steps allow for enrichment and/or isolation of fNRBCs without the need for pre-enrichment steps prior to filtration (e.g., density separation or aggregation of adult red blood cells using an aggregation reagent) and without the need for negative selection (e.g., negative immunoselection) to deplete one or more nucleated cell types other than fNRBCs, e.g., maternal lymphocytes, from the biological sample. Thus, in some embodiments, the methods of enriching for fNRBCs of the disclosure are performed in the absence of a pre-enrichment step prior to filtration and/or in the absence of a negative selection step.

5.3.1.Filtration

A filter of the disclosure can be any filter capable of separating nucleated cells from non-nucleated cells present in a biological sample. Suitable filters include filters comprised of a porous medium that permits components of a biological sample which are not of interest, (e.g., fluids, non-nucleated red blood cells, small particles, etc.) to pass through the porous medium. A pressure gradient can be established through the porous medium, whereby the upstream pressure exceeds the downstream pressure, thereby promoting passage of the fluid phase, as well as particulate matter not retained on the porous medium, through the porous medium. Matter unable to traverse the porous medium is retained on the porous medium. Most of this matter (e.g., nucleated cells including NRBCs) is selectively retained on the porous medium. While pressure gradients of varying degrees can be established through the porous medium, preferably the pressure is not so great as to damage the rare cells retained on the porous medium. Pressure can be applied, for example, by simply allowing a biological sample to flow through the filter under the force of gravity.

A porous medium can be fashioned from any appropriate substance, such as organic or inorganic material. Preferably, however, the porous medium comprises synthetic material such as a polymeric material, e.g., a polyamide, a polypropylene and/or a polyester having alipathic or aromatic groups. Examples of porous media suitable for use in the enrichment and isolation methods of the disclosure include those media described in U.S. Pat. Nos. 4,880,548, 4,925,572, and 6,544,751, U.S. Publication No. 2013/0130266, and PCT application publications WO 96/11738, WO 95/17236, WO 94/17894, the contents of each of which are incorporated herein by reference in their entirety. Such porous media are often employed as leukocyte reduction filters for donated blood and blood products, and a variety of such porous media are commercially available, such as, for example the Pall® Purecell Neo, Pall® RC-100 and RCXL2 leukocyte reduction filters, the Pall® SQ-40S Blood transfusion filter [symbol] filters, etc.

Most leukocyte reduction filters in use today can be described as “depth filters.” In a depth filter, various types of fibers, such as polyester and polypropylene, are woven into layers to create a “torturous path” for the blood cells to pass through. The average “pore size” of the woven fibers is quite large—on the order of 40 microns or more—to avoid trapping cells by mechanical properties such as size. Instead, the trapping mechanism appears to be primarily that of “adhesion” in which negatively charged leukocytes and other nucleated cells are attached or attracted to the fibers by Van der Waals and electrostatic forces. A fiber can be selected for its natural positive charge or can be coated to create very specific charge profiles and to ensure good hydrophilicity. An overall large pore size combined with the dense woven layers and charged fiber surfaces create a filter in which blood can pass through rapidly yet the individual cells follow a slower torturous path where they can adhere to the fibers.

After passing a biological sample through a filter (e.g., with or without one or more chases), a fNRBC-containing cell fraction can be collected from the filter. The fNRBC-containing cell fraction can be collected, for example, by passing an elution buffer through the filter in the reverse direction from the direction in which the biological sample was passed through the filter. An elution buffer can comprise, for example, a buffer of physiological pH such as a PBS buffer. The elution buffer can optionally comprise a charge neutralizing component, such as a dextran, to promote detachment of cells from the filter.

5.3.2. Positive Selection

A positive selection reagent of the disclosure can be any reagent that can be used to distinguish fNRBCs in a biological sample from at least one other type of cell in the sample.

A preferred approach for fNRBC enrichment is the use of positive immunoselection methods carried out in a fluid medium following filtration of a biological sample. Typically, the positive immunoselection methods utilize a positive immunoselective antibody. In certain aspects, a plurality of positive immunoselective antibodies are used in a positive immunoselection procedure.

To practice positive immunoselection, a positive immunoselective antibody is added to a sample, for example a fNRBC-containing cell fraction obtained by filtering a biological sample (e.g., maternal blood) through a filter such as a leukocyte reduction filter. The amount of antibody necessary to bind fNRBCs can be empirically determined by performing a test separation and analysis. The cells and antibody are incubated for a period of time sufficient for complexes to form, usually at least about 5 minutes, more usually at least about 10 minutes, and usually not more than one hour, more usually not more than about 30 minutes.

The sample may additionally be incubated with additional positive selecti [symbol] described herein, simultaneously or serially.

The cells can separated in accordance with the specific antibody preparation. Fluorochrome-labeled antibodies are useful for FACS separation, magnetic particles for immunomagnetic selection, particularly high gradient magnetic selection (HGMS), etc. Exemplary magnetic separation devices are described in WO 90/07380, PCT/US96/00953, and EP 0438520. Selection can also be performed using other automated methods, such as ultrafiltration or microfluidic separation. Selection can also be performed by micromanipulation, for example after identifying cells labeled with one or more positive selection reagents.

Accordingly, in some aspects, the present disclosure provides a method for preparing fNRBCs, comprising subjecting a sample comprising fNRBCs (e.g., a fNRBC-containing cell fraction obtained by a filtration step as described herein) to positive immunoselection, said positive immunoselection comprising the steps of: (a) contacting the sample with a positive immunoselective antibody in a fluid medium, wherein the positive immunoselective antibody selectively binds to fNRBCs relative to one or more other cell types in the sample; and (b) selecting cells bound to said positive immunoselective antibody.

5.3.3. Positive Selection Markers and Antibodies

Positive selection markers for fNRBCs include glycophorin A (also known as CD235a), “i” antigen, CD36, CD71, and nuclear markers. Where the downstream analysis permits cell fixation (e.g., FISH), fetal hemoglobin can be a positive selection marker.

Cells expressing the markers glycophorin A, “i” antigen, CD36, CD71 and fetal hemoglobin can be selected (e.g., sorted or enriched for) using antibodies against the markers.

In contrast to maternal erythrocytes, fNRBCs are nucleated and can be selected using nuclear dyes, such as Hoechst 33342, LDS751, TO-PRO, DC-Ruby, Cy5 and DAPI.

In some embodiments, fNRBCs are selected for using the monoclonal antibody 4B9. The hybridoma producing the antibody 4B9 is deposited at the Deutsche Sammlung von Mikroorganismen and Zelkulturen GmbH under accession number DSM ACC 2666 fNRBCs (see U.S. Pat. Nos. 7,858,757 B2 and 8,563,312 B2 of Hollmann et al.). In other embodiments, fNRBCs are selected for using an antibody that competes with 4B9 for binding to the surface of fNRBCs. By way of example, monoclonal antibody 4B8 competes with 4B9 for binding to fNRBCs (see US Pat. Nos. 7,858,757 B2 and 8,563,312 B2 of Hollmann et al.).

Further antibodies that bind to fNRBCs can be generated using the methods described in Hollmann et al. The ability to compete with 4B9 for binding to fNRBCs be tested using a competition assay. In one example of a competition assay, 4B9 antibody is used to isolate its target antigen (e.g., from fetal liver cells) and the target antigen is adhered onto a solid surface, e.g., a microwell plate. A mixture of sub-saturating amount of biotinylated and ur [symbol] candidate competing antibody (the “test” antibody) in serial dilution in ELISA buffer is added to wells and plates are incubated for 1 hour with gentle shaking. The plate is washed, HRP-conjugated Streptavidin diluted in ELISA buffer is added to each well and the plates incubated for 1 hour. Plates are washed and bound antibodies are detected by addition of substrate (e.g., TMB, Biofx Laboratories Inc., Owings Mills, Md.). The reaction is terminated by addition of stop buffer (e.g., Bio FX Stop Reagents, Biofx Laboratories Inc., Owings Mills, Md.) and the absorbance is measured at 650 nm using microplate reader (e.g., VERSAmax, Molecular Devices, Sunnyvale, Calif.). Alternatively, instead of isolating the antigen, whole fNRBCs can be used. In one approach, 1 microgram/ml of 4B9 conjugated to a first fluorescent dye (e.g., FITC) is added to microtiter wells containing 1×10⁵ fetal liver cells. The test antibody conjugated to a second fluorescent dye (e.g., phycoerythrin) is titrated at concentration from 10 microgram/ml to going down to 0.001 micrograms/ml (five 1 to 2 serial dilutions). Mean fluorescent intensities are measured for both antibodies. A test antibody is said to compete with 4B9 if the MFI of the reference antibody is reduced by at least 50% when the test antibody is added at same concentration as the reference antibody or at a lower concentration. In some embodiments, the MFI is reduced by at least 60%, at least 70% or at least 80%. Other formats for competition assays are known in the art and can be employed.

5.3.4. Antibody Labeling

Conveniently, the antibodies and nuclear stains used in the positive selection processes of the disclosure can be modified to permit selection and separation of the fNRBCs from other cells types. The modified antibodies can comprise any molecule or substance that allows sorting and detection, e.g., a magnetic bead or fluorochrome. In particular embodiments, the antibodies are coupled to a colorimetric molecule, a fluorescent moiety, a chemiluminescent moiety, an antigen, an enzyme, a detectable bead (such as a magnetic or electrodense (e.g., gold) bead), or a molecule that binds to another molecule (e.g., biotin or streptavidin)).

Fluorochromes can be used with a fluorescence activated cell sorter. Multi-color analyses can be employed with the FACS or in a combination of immunomagnetic separation and flow cytometry. Multi-color analysis is of interest for the separation of cells based on multiple surface antigens. Fluorochromes which find use in a multi-color analysis include phycobiliproteins, e.g., phycoerythrin and allophycocyanins; fluorescein and Texas red. A negative designation indicates that the level of staining is at or below the brightness of an isotype matched negative control. A dim designation indicates that the level of staining may be near the level of a negative stain, but may also be brighter than an isotype matched control. A positive immunoselective antibody of the disclosure preferably gives rise to a “bright” designation with respect to fNRBCs and a “negative” or “dim” designation with respect to one or more other cell types that can be present in a biological sample in which the fNR [symbol] present, such as maternal blood.

In one embodiment, an immunoselective antibody is directly or indirectly conjugated to a magnetic reagent, such as a superparamagnetic microparticle (microparticle). Direct conjugation to a magnetic particle is achieved by use of various chemical linking groups, as known in the art. The antibody can be coupled to the microparticles through side chain amino or sulfhydryl groups and heterofunctional cross-linking reagents. A large number of heterofunctional compounds are available for linking to entities. A preferred linking group is 3-(2-pyridyidithio)propionic acid N-hydroxysuccinimide ester (SPDP) or 4-(N-maleimidomethyl)-cyclohexane-1-carboxylic acid N-hydroxysuccinimide ester (SMCC) with a reactive sulfhydryl group on the antibody and a reactive amino group on the magnetic particle.

Alternatively, an immunoselective antibody can be indirectly coupled to the magnetic particles. The antibody can be directly conjugated to a hapten, and hapten-specific, second stage antibodies are conjugated to the particles. Suitable haptens include digoxin, digoxigenin, FITC, dinitrophenyl, nitrophenyl, avidin, biotin, etc. Methods for conjugation of the hapten to a protein are known in the art, and kits for such conjugations are commercially available.

Fluorescent labels may include rhodamine, lanthanide phosphors, fluorescein and its derivatives, fluorochrome, GFP (GFP for “Green Fluorescent Protein”), dansyl, umbelliferone, phycoerythrin, phycocyanin, allophycocyanin, o-phthaldehyde, and fluorescamine.

Enzymatic labels may include horseradish peroxidase, β galactosidase, luciferase, alkaline phosphatase, glucose-6-phosphate dehydrogenase (“G6PDH”), alpha-D-galactosidase, glucose oxidase, glucose amylase, carbonic anhydrase, acetylcholinesterase, lysozyme, malate dehydrogenase and peroxidase.

Chemiluminescent labels or chemiluminescers, such as isoluminol, luminol and the dioxetanes.

Other detectable moieties include molecules such as biotin, digoxygenin or 5-bromodeoxyuridine.

In some embodiments, an immunoselective antibody is not directly modified for selection or detection but used as a primary antibody. A secondary antibody that is modified, e.g., by attachment to a magnetic bead or a fluorescent dye, can be used to select for or detect cells bound to the primary antibody.

5.3.5. Selection Techniques

An immunoselection step can utilize, for example, magnetic separation, e.g., using antibody-coated magnetic beads, or flow cytometry. Flow cytometric techniques can provide accurate separation via the use of, e.g., fluorescence activated cell sorters, which can have varying degrees of sophistication, such as multiple color channels, low angle an [symbol] scattering detecting channels, impedance channels, etc. In various aspects, MACs, but not FACS, is used to enrich for fNRBCs.

Conveniently, positive selection reagents can be conjugated with labels, e.g., magnetic beads and fluorochromes, to allow for ease of separation of the fNRBCs from other cells types. Fluorochromes can be used with a fluorescence activated cell sorter. Multi-color analyses can be employed with the FACS or in a combination of immunomagnetic separation and flow cytometry. Multi-color analysis is of interest for the separation of cells based on multiple surface antigens. Fluorochromes which find use in a multi-color analysis include phycobiliproteins, e.g., phycoerythrin and allophycocyanins; fluorescein and Texas red. A negative designation indicates that the level of staining is at or below the brightness of an isotype matched negative control. A dim designation indicates that the level of staining may be near the level of a negative stain, but may also be brighter than an isotype matched control. A positive immunoselective antibody of the disclosure preferably gives rise to a “bright” designation with respect to fNRBCs and a “negative” or “dim” designation with respect to one or more (and in some embodiments all) other cell types that can be present in a biological sample in which the fNRBCs are present, such as maternal blood.

In one embodiment, an immunoselective antibody is directly or indirectly conjugated to a magnetic reagent, such as a superparamagnetic microparticle (microparticle). Direct conjugation to a magnetic particle is achieved by use of various chemical linking groups, as known in the art. The antibody can be coupled to the microparticles through side chain amino or sulfhydryl groups and heterofunctional cross-linking reagents. A large number of heterofunctional compounds are available for linking to entities. A preferred linking group is 3-(2-pyridyidithio)propionic acid N-hydroxysuccinimide ester (SPDP) or 4-(N-maleimidomethyl)-cyclohexane-1-carboxylic acid N-hydroxysuccinimide ester (SMCC) with a reactive sulfhydryl group on the antibody and a reactive amino group on the magnetic particle.

Alternatively, an immunoselective antibody is indirectly coupled to the magnetic particles. The antibody is directly conjugated to a hapten, and hapten-specific, second stage antibodies are conjugated to the particles. Suitable haptens include digoxin, digoxigenin, FITC, dinitrophenyl, nitrophenyl, avidin, biotin, etc. Methods for conjugation of the hapten to a protein are known in the art, and kits for such conjugations are commercially available.

To practice positive immunoselection, a positive immunoselective antibody is added to a biological sample. The amount of antibody necessary to bind NRBCs can be empirically determined by performing a test separation and analysis. The cells and antibody are incubated for a period of time sufficient for complexes to form, usually at least about 5 minutes, more usually at least about 10 minutes, and usually not more than one hour, more usually not more than about 30 minutes.

The biological sample may additionally be incubated with additional posit [symbol] immunoselective antibodies as described herein. The labeled cells are separated in accordance with the specific antibody preparation. Fluorochrome-labeled antibodies are useful for FACS separation, magnetic particles for immunomagnetic selection, particularly high gradient magnetic selection (HGMS), etc. Exemplary magnetic separation devices are described in WO 90/07380, PCT/US96/00953, and EP 438,520.

The positive immunoselection can be performed using other automated methods, such as ultrafiltration or microfluidic separation.

The methods of the disclosure are preferably performed with one or more positive immunoselection steps in a fluid phase and one or more positive immunoselective antibodies in soluble format, i.e., not immobilized on a solid surface. The methods of the disclosure can be adapted to incorporate one or more steps in which a positive and/or immunoselective antibody is bound to a solid surface. Immobilizing 4B9 on a solid surface for cell capture is, for example, described in U.S. application Ser. No. 13/295,532, filed Nov. 14, 2011 and published as US 2013/0122492 on May 16, 2013, the contents of which are incorporated by reference in their entireties herein.

5.4. Downstream Isolation Techniques

Following filtration (and positive selection, if performed), fNRBCs can be isolated by capture on a solid surface (e.g., with a positive immunoselective antibody such as 4B9 or a secondary antibody to capture positive immunoselective antibody-bound cells) or by a physical technique such as micromanipulation.

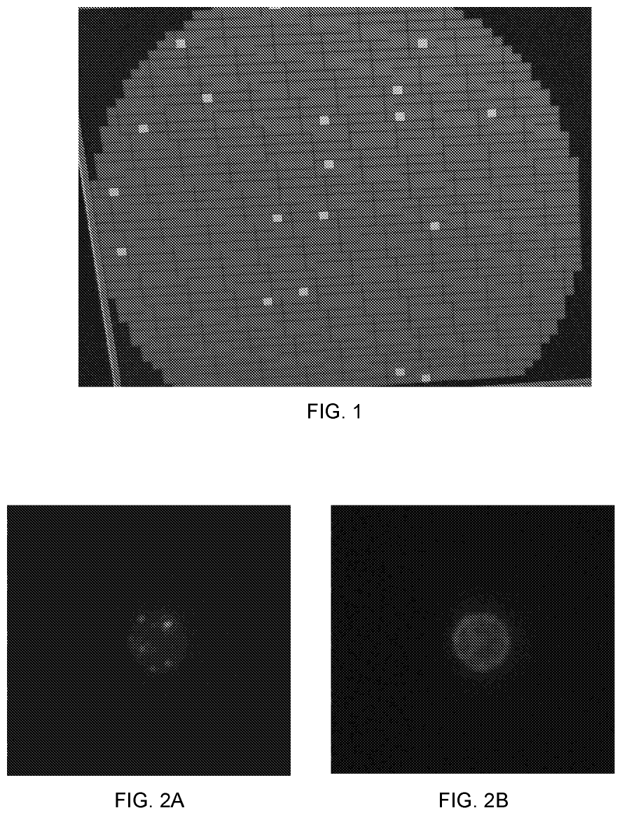

A detectable moiety attached to a positive immunoselective antibody can be used to identify and isolate the fetal NRBCs. Preferably, multiple positive selection reagents are used to identify and isolate fNRBCs. For example, fNRBCs can be labeled with 4B9, an anti-CD235a antibody, and a nuclear stain, and the cells which are “triple positive” for all three reagents can be identified and isolated. Identification of labeled cells can be done manually, for example by examining individual cells under a fluorescence microscope, or by an automated process, for example by imaging a substrate containing a population of labeled cells with a fluorescence microscope and using software (e.g., NIS-Elements from Nikon) to identify labeled cells (see, e.g., FIG. 1). When using an automated process, the cells are preferably placed on a smooth substrate (such as a glass petri dish) so that the cells are generally in the same plane. When cells are in the same plane, a large number of cells can be analyzed accurately and relatively quickly, as the fluorescence microscope will not lose focus as it images different cells on the substrate. Glass petri dishes, which are typically used for cell culture, are readily available and have been found to allow quick, accurate analysis of a large number of cells. Cells identified as fNRBCs can then be isolated, for example, by micromanipulation.

Micromanipulation may be performed under a microscope or through oth [symbol] enhancement or assistance. Micromanipulation may be performed through an automated process or by using manual micromanipulation equipment. For instance, micromanipulation may select or isolate a single fNRBC or multiple fNRBCs. For example, groups of 1, 5, 10 or 20 cells may be isolated by micromanipulation and placed in individual sample tubes of 1, 5, 10 or 20 cells. In some embodiments, one, two, three, four or five groups of 1-20 cells, e.g., 1-5 cells, 1-10 cells, 5-20 cells, or 5-10 cells are isolated by micromanipulation.

In some embodiments, the isolation techniques (e.g., micromanipulation) can take advantage of the fluorescent labels utilized to enrich the cells, the presence of hemoglobin in the fNRBCs (detectable by a Soret band filter) and fNRBC morphological features (Huang et al., 2011, J Cell Biochem. 112:1475-85; Choolani et al., 2003, Mol Hum Repro 9:227-35).

5.5. Populations of fNRBCs

The present disclosure further provides preparations of fNRBCs prepared or obtainable by the methods described herein. Exemplary preparations include populations of cells comprising fNRBCs.

In some embodiments, the populations of cells are obtained or obtainable from maternal blood, e.g., maternal blood drawn between about 4 and about 38 weeks of pregnancy or between about 6 weeks and about 20 weeks of pregnancy, by any of the exemplary protocols described in Section 6. In some embodiments, the enrichment entails filtration, with or without a subsequent MACS step for positive enrichment. Preferably, the enrichment includes a MACS step following filtration to further reduce the number of maternal cells in the population.

The fNRBCs can be primitive fNRBCs, definitive fNRBCs, or a mixture of both. In some embodiments, the ratio of primitive and definitive fNRBCs is a ratio found in maternal blood about 6 weeks to about 20 weeks of gestation. The fNRBCs can be bound to antibody, e.g., one or more of the positive immunoselective antibodies described herein, or free of antibody. Such antibody-free fNRBCs can be prepared, for example, by stripping a positive immunoselective antibody from the cells.

When the fNRBCs are prepared from a maternal blood sample, the remaining cells in the population are typically one or more cell types present in maternal blood during gestation.

5.6. Validation of fNRBCs

Genetic fingerprinting methods that involve, for example, generating a genetic profile using Short Tandem Repeat (STR) analysis, Restriction Fragment Length Polymorphism (RFLP) analysis or Single Nucleotide Polymorphism (SNP) analysis can be used to validate an fNRBC or fNRBCs isolated by the methods described herein as a fetal cell(s). By comparing the profile generated from the isolated cell(s) to a profile generated from maternal and optionally, paternal cells, the identity of the isolated cell(s) as a fetal cell(s) can be verified. [symbol] generating genetic profiles are commercially available. For example, the PowerFlex® Fusion STR kit from Promega and the Genome-Wide Human SNP Array 6.0 from Affymetrix can be used to generate STR and SNP profiles, respectively, which can be used to validate the identity of fNRBCs. In some embodiments, whole genome amplification (WGA) is used to increase the amount of genetic material available for analysis.

5.7. Downstream Analysis

The preparations can be used in fetal diagnostic testing, e.g., for determining the presence of a fetal abnormality. Examples of abnormalities that can be tested for include trisomy 13, trisomy 18, trisomy 21, Down syndrome, neuropathy with liability to pressure palsies, neurofibromatosis, Alagille syndrome, achondroplasia, Huntington's disease, alpha-mannosidosis, beta-mannosidosis, metachromatic leucodystrophy, von Recklinghausen's disease, tuberous sclerosis complex, myotonic dystrophy, cystic fibrosis, sickle cell disease, Tay-Sachs disease, beta-thalassemia, mucopolysaccharidoses, phenylketonuria, citrullinuria, galactosemia, galactokinase and galactose 4-epimerase deficiency, adenine phosphoribosyl, transferase deficiency, methylmalonic acidurias, proprionic acidemia, Farber's disease, fucosidosis, gangliosidoses, gaucher's disease, I cell disease, mucolipidosis III, Niemann-Pick disease, sialidosis, Wolman's disease, Zellweger syndrome, cystinosis, factor X deficiency, ataxia telangiectasia, Bloom's syndrome, Robert's syndrome, xeroderma pigmentosum, fragile (X) syndrome, sex chromosome aneuploidy, Klinefelter's Syndrome, Turner's syndrome, XXX syndrome, steroid sulfatase deficiency, microphthalmia with linear skin defects, Pelizaeus-Merzbacher disease, testis-determining factor on Y, ornithine carbamoyl transferase deficiency, glucose 6-phosphate dehydrogenase deficiency, Lesch-Nyhan syndrome, Anderson-Fabry disease, hemophilia A, hemophilia B, Duchenne type muscular dystrophy, Becker type muscular dystrophy, dup(17)(p11.2p11.2) syndrome, 16p11.2 deletion, 16p11.2 duplication, Mitochondrial defect, dup(22)(q11.2q11.2) syndrome, Cat eye syndrome, Cri-du-chat syndrome, Wolf-Hirschhorn syndrome, Williams-Beuren syndrome, Charcot-Marie-Tooth disease, chromosome rearrangements, chromosome deletions, Smith-Magenis syndrome, Velocardiofacial syndrome, DiGeorge syndrome, 1p36 deletion, Prader-Willi syndrome, Azospermia (factor a), Azospermia (factor b), Azospermia (factor c), spina bifida, anencephaly, neural tube defect, microcephaly, hydrocephaly, renal agenesis, Kallmann syndrome, Adrenal hypoplasia, Angelman syndrome, cystic kidney, cystic hygroma, fetal hydrops, exomphalos and gastroschisis, diaphragmatic hernia, duodenal atresia, skeletal dysplasia, cleft lip, cleft palate, argininosuccinicaciduria, Krabbe's disease, homocystinuria, maple syrup urine disease, 3-methylcrotonyl coenzyme A, carboxylase deficiency, Glycogenoses, adrenal hyperplasia, hypophosphatasia, placental steroid sulphatase deficiency, severe combined immunodeficiency syndrome, T-cell immunodeficiency, Ehlers-Danlos syndrome, osteogenesis imperfect, adult polycystic kidney disease, Fanconi's anemia, epidermolysis bullosa syndromes, ectodermal dysplasia, congenital nephrosis (Finnish type) and multiple endocrine neoplasia.

The diagnostic assay can be a nucleic acid (e.g., DNA or RNA) assay, a protein (e.g., antibody-based) assay, or a histology assay, or a combination thereof. Examples of DNA assays include FISH, PCR, DNA sequencing, and rolling cycle replication product assays. Examples of RNA assays include RT-PCR assay and FISH assays. To facilitate access to the nucleic acid, the fNRBCs can be lysed or permeabilized prior to carrying out the diagnostic test. The DNA, RNA and protein assays can be performed on a microarray. Illustrative methods are described below.

In some embodiments, single cells or groups of cells (e.g., two to four or more) can be amplified by whole genome amplification (WGA) to provide sufficient nucleic acid for analysis. Groups of cells (e.g., containing 5 or more fetal NRBCs) can be analyzed without the use of whole genome amplification (WGA). WGA refers to amplification of the entire genome of a cell or group of cells of an individual. For example, a whole genome can be amplified using the genetic material of a single cell (i.e., single cell whole genome amplification (SCWGA)).

Chromosomal abnormalities, single gene abnormalities, allelic variants and single nucleotide polymorphisms (SNPs) are detectable using the chromosomes or nucleic acid from lysed fetal NRBCs produced by the methods of the present disclosure by any of a variety of methods, including fluorescence in situ hybridization (FISH), polymerase chain reaction (PCR), multiple annealing and looping based amplification cycles (MALBAC), restriction fragment length polymorphism (RFLP) analysis, DNA sequencing and imaging of labeled rolling circle amplification products. The PCR technique can be a simple PCR amplification technique or a quantitative PCR, a real-time PCR or a reverse transcriptase PCR technique. Other useful genetic analysis techniques include array comparative genomic hybridization (CGH) and analysis in a DNA microarray. For instance, the fetal NRBCs can be analyzed in a prenatal chromosomal microarray.

A haplotype is a combination of alleles that occur together and at adjacent locations on a chromosome. A haplotype may be found on a single locus or on several loci. Haplotypes may occur throughout an entire chromosome. Haplotypes may include any number of recombination events. A haplotype may also refer to a set of associated single nucleotide polymorphisms.

A single nucleotide polymorphism (SNP) occurs where there is a variation from a normal (e.g., wild type) nucleotide sequence in a single nucleotide (e.g., A, T, C or G). For example, a single nucleotide polymorphism may result in an allelic variant. A given allele may be defined by a single nucleotide polymorphism or by multiple nucleotide changes.

Restriction Fragment Length Polymorphisms (RFLPs) are differences in homologous sequences of DNA. They may be detected by differences in fragment lengths found after digestion of DNA using a particular restriction endonuclease or combination of r [symbol] endonucleases. RFLP may be determined by gel electrophoresis or southern blo [symbol] s.

Fluorescence in situ hybridization (FISH) is performed by binding fluorescent probes to a portion of a fixed nucleic acid sequence complement to that of the fluorescent probe. FISH can be used to fluorescently tag a target nucleic acid sequence in RNA or DNA at the specific position where a nucleic acid sequence occurs within a larger nucleic acid sequence. For example, FISH may be used to tag a target sequence on a chromosome. The fluorescent probe may be viewed using fluorescence microscopy.

PCR is used to amplify one or more copies (i.e., amplicons) of a particular nucleic acid sequence by using two primers. PCR methods are readily available and are commonly used to diagnose hereditary diseases. Non-PCR based methods can also be used to amplify a particular nucleic acid sequence for analysis, for example, rolling circle amplification (RCA) (see, e.g., Dahl et al., 2018, Nature Scientific Reports, 8:4549).

Quantitative PCR (qPCR) is based on a polymerase chain reaction (PCR) and is used to both amplify and simultaneously quantify the total number of copies or the relative number of copies of a nucleic acid sequence. One example of qPCR is Real-Time PCR. In Real-Time PCR, the number or relative number of nucleic acid copies resulting from PCR are detected in real time. The number or relative number of copies produced by qPCR may be detected and quantified using a signal generated by fluorescent dyes.

Reverse Transcription Polymerase Chain Reaction (RT-PCR) is a method which can be used to detect RNA molecules or to determine the expression levels of a specific RNA sequence (e.g., mRNA) by transcribing the RNA molecule(s) into DNA copies (cDNA) and amplifying the DNA. RT-PCR may be performed by a one-step or two-step process.

Array Comparative Genomic Hybridization (array CGH) is a microarray technique used to determine chromosome copy number variations that occur on a genome-wide scale. Array CGH compares a test genome with a normal (e.g., wild type) genome to detect even relatively small (e.g., 200 base pairs) structural variations. For example, array CGH may detect deletions, amplifications, breakpoints or aneuploidy. Array CGH may also be used to detect a predisposition for developing a cancer.

Multiple Annealing and Looping Based Amplification Cycles (MALBAC) is a whole genome amplification method. MALBAC can be used for single cell, whole genome amplification. MALBAC can be used to amplify a genome in a quasi-linear fashion and avoid preferential amplification of certain DNA sequences. In MALBAC, amplicons may have complementary ends, which form loops in the amplicon and therefore prevent exponential copying of the amplicon. Amplicon loops may prevent amplification bias. MALBAC can be applied to diagnosing fetal abnormalities using a single fNRBC, or may be used [symbol] fetal predisposition for developing a cancer using a single fNRBC.

Next Generation Sequencing (NGS) is a group of high-throughput sequencing technologies that can be used for detecting a fetal abnormality. NGS (e.g., massively parallel sequencing) uses a cell sample as small as a single cell to sequence large stretches of nucleic acid sequences or an entire genome. For example, in NGS many relatively small nucleic acid sequences may be simultaneously sequenced from a genomic DNA (gDNA) sample from a library of small segments (i.e., reads). The reads can then be reassembled to identify a large nucleic acid sequence or a complete nucleic acid sequence of a chromosome. For instance, in NGS, as many as 500,000 sequencing operations may be run in parallel. NGS is a form of single cell, whole genome amplification (WGA). For instance, MALBAC may be used for NGS when followed by traditional PCR.

Massively Parallel Signature Sequencing (MPSS) is one example of an NGS. MPSS identifies mRNA transcripts from 17-20 base pair signature primer sequences. MPSS can be utilized to both identify and quantify mRNA transcripts in a sample (Brenner et al., 2000, Nature biotechnology 18(6): 630-634, 2000).

Polony Sequencing is another example of NGS. Polony sequencing can be used to read millions of immobilized DNA sequences in parallel. Polony sequencing is a multiplex sequencing technique that has been found to be extremely accurate (low error rate) (Shendure et al., 2004. Nature Reviews Genetics 5(5): 335-344, 2004; Shendure et al., 2008, Nature Biotech 26(10): 1135-1145).

454 Pyrosequencing is another example of NGS. 454 pyrosequencing utilizes luciferase to detect individual nucleotides added to a nascent DNA. 454 pyrosequencing amplifies DNA contained in droplets of water in an oil solution. Each droplet of water contains one DNA template attached to a primer-coated bead (Vera et al., 2008, Molecular Ecology 17(7): 1636-1647).

Illumina Sequencing is another example of NGS. In Illumina Sequencing DNA molecules and primers are attached to a slide. The DNA molecules are amplified by a polymerase and DNA colonies (DNA clusters) are formed (Shendure et al., 2008, Nature Biotech 26(10): 1135-1145; Meyer et al., 2010, Cold Spr Hbr Protocols 2010(6): pdb-prot 5448).

Sequencing by Oligonucleotide Ligation and Detection (SOLiD Sequencing) is another example of NGS. SOLiD sequencing is a method of sequencing by ligation. SOLiD sequencing randomly generates thousands of small sequence reads simultaneously and immobilizes the DNA fragments on a solid support for sequencing (Shendure et al., 2008, Nature Biotech 26(10): 1135-1145; Meyer et al., 2009, New Biotechnology 25(4):195-203).

Ion Torrent Semiconductor Sequencing is another example of NGS. Ion [symbol] Semiconductor Sequencing is a sequencing-by-synthesis method that detects hydrogen ions released during DNA polymerization. A deoxyribonucleotide triphosphate is introduced into a microwell containing a template DNA strand to be sequenced. When the dNTP is complementary to a leading template nucleotide, the dNTP is incorporated into the complementary DNA strand and a hydrogen ion is released (Quail et al., 2012, BMC Genomics 13(1): 341).

DNA Nanoball Sequencing is another example of NGS. DNA Nanoball Sequencing can be used to determine an entire genomic sequence of an organism, such as, for instance, a newly discovered organism. Small fragments of genomic DNA are amplified using rolling circle replication to form DNA nanoballs. DNA sequences can then be ligated by using fluorescent probes as guides (Ansorge et al., 2009, New Biotechnology 25(4): 195-203; Drmanac et al., 2010, Science 327(5961):78-81).

Heliscope Single Molecule Sequencing is another example of NGS. Heliscope Single Molecule Sequencing is a direct-sequencing approach that does not require ligation or PCR amplification. DNA is sheared, tailed with a poly-A tail and then hybridized to the surface of a flow cell with oligo(dT). Billions of molecules may be then sequenced in parallel (Pushkarev et al., 2009, Nature Biotechnology 27(9): 847-850).

Single Molecule Real Time (SMRT) Sequencing is another example of NGS. SMRT sequencing is a sequencing-by-synthesis approach. DNA is synthesized in small well-like containers called zero-mode wave-guides (ZMWs). Unmodified polymerases attached to the bottom of the ZMWs are used to sequence the DNA along with fluorescently labeled nucleotides which flow freely in the solution. Fluorescent labels are detached from the nucleotides as the nucleotide is incorporated into the DNA strand (Flusberg et al., 2010, Nature methods 7(6): 461-465).

Ultra-Deep Sequencing refers to the number of times that a nucleic acid sequence is determined from many template copies. Ultra-Deep Sequencing may be used to identify rare genetic mutations by amplifying a relatively small target nucleic acid sequence which may contain a rare mutation.

DNA Microarray can be used to measure the expression levels of multiple genes simultaneously. DNA Microarray can also be used to genotype multiple regions of a genome. For example, Prenatal Chromosomal Microarray (CMA)—can be used to detect copy-number variations, such as aneuploidies in a chromosome. Prenatal CMA may detect deletions or duplications of all or part of a chromosome.

In certain aspects, a single fNRBC or a small group of fNRBCs can be subject to DNA fingerprinting, for example on a SNP microarray using the principles described by Treff et al., 2010, Fertility and Sterility 94(2):477-484, which is incorporated by reference he [symbol] entirety. The SNP microarrays to be used in these methods are preferably genome-wide SNP arrays. In various embodiments, the SNP fingerprint comprises at least 50,000, at least 100,000, at least 150,000, at least 200,000 or at least 250,000 SNPs. The SNP fingerprint can be generated from a single microarray or multiple microarrays. Using comparative DNA fingerprinting, a fNRBC can be distinguished from a maternal cell. In preferred embodiment, the determination of a match with the maternal cell (e.g., that the cell under examination is a maternal, rather than fetal, cell) is based on at least 1,000, more preferably at least 1,500 and yet more preferably at least 2,000 informative SNPs. The maternal fingerprint can be based on a historical maternal sample or a maternal sample run in parallel with the fNRBC. The DNA fingerprinting can be preceded by WGA of the fNRBC and optionally the maternal sample. The SNP fingerprint can also be used to fetal abnormalities or other characteristics. Microarrays can be adapted to include a combination of SNPs and markers of fetal characteristics and/or possible fetal cell abnormalities, such as those described above. In particular embodiments, the microarrays include at least 5, at least 10, at least 15, at least 20, at least 30 or at least 50 markers of possible fetal cell abnormalities and/or markers of fetal sex, such as Y chromosome markers.

5.8. Kits

The present disclosure further provides kits comprising one or more antibodies useful in the positive immunoselection methods of the disclosure, such the antibodies described in Section 5.3.3 above. In some embodiments, the kits comprise the antibody 4B9. The antibodies can be attached to a detectable moiety, e.g., biotin or a fluorescent moiety. If the antibodies are biotinylated, the kit can also include an avidin-conjugated detection reagent (i.e., antibody).

The kits can also include a nuclear stain for better selection of fNRBCs.

The kits can also include a filter, for example filter as described in Section 5.3.1.

Buffers and the like useful for using the antibodies for enrichment of fNRBCs are well-known in the art and may be prepared by the end-user or provided as a component of the kit. The kit may also include a solid support containing positive- and negative-control tissue samples, e.g., fetal liver cells as positive controls and/or adult blood or cellular components of adult blood as negative controls.

The kits can also include one or more reagents suitable for fetal cell diagnostics, such as reagents suitable for carrying out the diagnostic methods described in Section 5.7 above. In an exemplary embodiment, the reagents include primers, e.g., for PCR or sequencing, and/or optionally probes, e.g., for detection of fetal cell abnormalities.

### 6. EXEMPLARY PROTOCOLS

Various combinations of the filtration protocol of Section 6.1 and the positive selection protocols of Section 6.2 can be used to enrich NRBCs from a sample comprising fNRBCs and maternal cells, e.g., maternal blood. Following enrichment, the enriched fNRBCs can be subject to fluorescent staining, for example as described in Section 6.3, for further analysis. For example, the following combinations of the protocols are within the scope of the disclosure.

Combination #1: filtration protocol #1 and positive selection protocol #1.

Combination #2: filtration protocol #1 and positive selection protocol #2.

Combination #3: filtration protocol #2 and positive selection protocol #3.

Combination #4: combination #1 followed by staining protocol #1.

Combination #5: combination #1 followed by staining protocol #2.

Combination #6: combination #2 followed by staining protocol #1.

Combination #7: combination #2 followed by staining protocol #2.

Combination #8: combination #3 followed by staining protocol #1.

Combination #9: combination #3 followed by staining protocol #2.

Combination #10: filtration protocol #1 followed by staining protocol #1.

Combination #11: filtration protocol #1 followed by staining protocol #2.

6.1. Filtration

The following exemplary positive filtration protocol #1 is suitable for use in the methods of the disclosure:
1. Starting with a sample of maternal blood, dilute the sample with a buffer such as PBS. For example, the sample can be diluted with an equal volume of a PBS buffer.
2. Filter the sample through a leukocyte reduction filter, e.g., a Pall Purecell® Neo filter. The sample can be filtered, by example, by positioning a blood bag containing the sample above the filter, connecting the blood bag to the filter with tubing, and allowing the sample to flow through the filter under the force of gravity.
3. Optionally perform one or more chases, e.g., three chases, with a buffer such as a PBS buffer.
4. Collect the cells remaining on the filter by flushing a buffer, such as a PBS buffer, through the filter in the reverse direction and collecting the buffer, which now contains nucleated cells. The PBS buffer can be forced through the filter under pressure, for example, pressure applied by a syringe.
5. Optionally repeat step 4.

6.2. Positive Selection

In some embodiments of the present disclosure, a sample comprising fNRBCs and maternal cells is subject to positive selection using the antibody 4B9.

6.2.1. Positive Selection Protocol #1

The following exemplary positive selection protocol #1 is suitable for use in the methods of the disclosure:
1. If starting with a suspension comprising fNRBCs obtained by filtration protocol #1, centrifuge the suspension to pellet cells, e.g., at 450×g for 10 minutes, and aspirate the supernatant completely. If starting with a cell pellet, begin at step 2.
2. Resuspend the pellet in a MACS running buffer.
3. Add an FcR blocking reagent, e.g., FcR Blocking Reagent (Miltenyi Biotec), and mix well. The FcR blocking reagent blocks non-specific Fc receptor-mediated antibody binding.
4. Add unconjugated 4B9; incubate to allow the unconjugated 4B9 to bind to fNRBCs.
5. Add MACS running buffer to the sample; centrifuge to pellet the cells, e.g., at 300×g for 6 minutes.
6. Aspirate the supernatant completely.
7. Add MACS running buffer to the sample and centrifuge the sample to pellet the cells, e.g., at 300×g for 6 minutes.
8. Aspirate the supernatant completely.
9. Resuspend the pellet in a MACS running buffer.
10. Add an FcR blocking reagent to the sample.
11. Add anti-IgM microbeads to the sample; incubate to allow microbeads to bind to 4B9.
12. Add MACS running buffer to the sample and mix. Then, centrifuge the sample to pellet the cells, e.g., at 300×g for 6 minutes. Aspirate the supernatant completely.
13. Add MACS running buffer to the sample.
14. Magnetically sort the cells using a MACS column according to manufacturer instructions to obtain a 4B9 positive fraction and a 4B9 negative fraction.

Steps 5-8 remove unbound 4B9 from the sample. Washing step 12 removes unbound anti-IgM microbeads from the sample.

6.2.2. Positive Selection Protocol #2

Positive selection protocol #1 modified by replacing unconjugated-4B9 with biotinylated 4B9 and replacing anti-IgM microbeads with anti-biotin microbeads provides positive selection protocol #2.

6.2.3. Positive Selection Protocol #3

4B9⁺ cells are selected by incubating with unconjugated 4B9, washing to remove unbound 4B9 antibody, binding the 4B9 coated cells with goat-anti-mouse-IgM microbeads, and then washing, resuspending and centrifuging the resulting cells. Following centrifugation, the supernatant is discarded and the pellet resuspended in a buffer such as PBS.

6.3. Staining

In some embodiments of the present disclosure, a sample comprising fNRBCs prepared according to the disclosure is fluorescently stained to allow for visualization and picking of isolated fNRBCs.

6.3.1. Staining Protocol #1

The following exemplary staining protocol #1 can be used to fluorescently stain a sample comprising fNRBCs:
1. Centrifuge (i) the 4B9 positive fraction prepared according to any one of combinations #1-3, above, or (ii) a fNRBC-containing cell fraction from filtration protocol #1 that has been incubated with 4B9, to pellet the cells, e.g., at 300×g for 6 minutes.
2. Aspirate the supernatant.
3. Add a fluorescent master mix to the sample and incubate, where the master mix contains a suitable labeled marker for detecting fNRBCs enriched according to any of the protocols described above, e.g., streptavidin Alexa 488 and/or goat anti-mouse IgM Alexa 488, anti-CD235a-PE antibody, and a nuclear marker, e.g., DC-Ruby.
4. Add a buffer to the sample, e.g., 1× PBS, to wash the cells.
5. Centrifuge the sample to pellet the cells, e.g., at 300×g for 5 minutes.
6. Aspirate the supernatant.
7. Resuspend the pellet in an appropriate buffer, e.g., 1× PBS.

6.3.2. Staining Protocol #2

The following exemplary staining protocol #2 can also be used to fluorescently stain a sample comprising fNRBCs:
1. Centrifuge (i) the 4B9 positive fraction prepared according to any one of combinations #1-3, above, or (ii) a fNRBC-containing cell fraction from filtration protocol #1 that has been incubated with 4B9, to pellet the cells, e.g., at 300×g for 6 minutes.
2. Aspirate the supernatant.
3. Add a fluorescent master mix to the sample and incubate, where the master mix contains a suitable labeled marker for detecting fNRBCs enriched according to any of the protocols described above, e.g., streptavidin Alexa 488 and/or goat a [symbol] Alexa 488, and anti-CD235a-PE antibody.
4. Add a buffer to the sample, e.g., 1× PBS, to wash the cells.
5. Centrifuge the sample to pellet the cells, e.g., at 300× g for 5 minutes.
6. Aspirate the supernatant.
7. Resuspend the pellet in an appropriate buffer, e.g., 1× PBS.
8. Add a nuclear stain, e.g., a Hoechst stain and mix.

Appropriate volumes and concentrations of reagents, temperatures, mixing times, centrifugation times, centrifugation forces, and specific reagents used in the above protocols can be selected by those having ordinary skill in the art. Similarly, persons having skill in the art will appreciate that washing steps can be added or omitted from the above protocols without changing the basic operation of the protocols.

6.4. Preparation for Downstream Analysis

Original biological samples containing fNRBCs or samples enriched for fNRBCs by any of the method steps described above, can be subject to further processing to enrich or isolate fNRBCs.

fNRBCs can be isolated by methods such as micromanipulation. Using micromanipulation techniques known in the art or described in the Section 6.5 below, individual fNRBCs can be picked and isolated.

Following enrichment, the cells can be subject to downstream analysis, for example Short Tandem Repeat (STR) analysis of their genomic DNA, DNA fingerprinting, chromosome copy number analysis, and/or other methods for verification of fetal cell identity, diagnosis of fetal abnormality or disease, and testing of fetal characteristics.

6.5. Cell Picking By Micromanipulation

For isolation of cells a commercial micromanipulator can be mounted onto an inverse phase contrast microscope. The microscope can equipped with various objectives, fluorescent filters, a camera, monitor, and joystick operated micromanipulator platform. Micromanipulation is composed in three linear axes—X, Y, and Z directions.

Cells fluorescently stained with various positive selection reagents are placed onto a suitable surface, e.g., a microscope slide, a glass bottom petri dish, or a plate (e.g., a Nunc OmniTray single-well plate, VWR catalog number 242811) and isolated with a sterile capillary tube with a diameter of the opening on the capillary tip configured to the size of the fNRBCs. The fluorescent stains can correspond to one or more antibodies that recognize fetal cells, for example selected from 4B9 (Zimmermann et al., 2013, Exp Cell Res 319:2700-2707), anti-CD34, anti-CD71, anti-glycophorin-A (anti-CD235a), and anti-i-antigen (Huang et al., 2011, J Cell Biochem. 112:1475-85; Choolani et al., 2003, Mol Hum Repro 9:227-35; Ca [symbol] 2012, Clin Genet. 82(2):131-9). If the cells are fixed, e.g., in order to perform FISH, anti-epsilon globin, reportedly a highly specific primitive fetal erythroblast identifier (Choolani et al., 2003, Mol Hum Repro 9:227-35; Choolani et al., 2001, Blood 98:554-7), can be used. Nuclear stains can also be used. In some embodiments, cells are stained with 4B9, an anti-CD235a antibody, and a nuclear stain.

Each positive selection reagent used during the fluorescent staining step(s) corresponds to its own specific fluorescent filter on the microscope and visualized either through the eye piece and/or monitor depending on the wavelengths. An automated system comprising a fluorescence microscope and software (e.g., NIS-Elements from Nikon) can be used to automatically image a sample to identify cells stained with the positive selection reagent(s). For example, when the positive selection reagents include 4B9, an anti-CD235a antibody, and a nuclear stain, the software can be used to identify “triple positive” cells stained with all three reagents (see, e.g., FIG. 1). A threshold at which the signal for each label is considered positive can be set prior to scanning a plate and can vary between runs, for example due to variations in staining efficiency. Thresholds can be set, for example, by manually identifying a fNRBC and selecting thresholds based upon the signal intensity of each label observed for the manually identified fNRBC.

In addition to fluorescent markers, selection criteria for fNRBCs can be hemoglobin content (detectable by a Soret filter) and morphological features. Primitive fNRBCs have distinguishing morphological features of having a high cytoplasmic to nuclear ratio and a comparatively larger size (Huang et al., 2011, J Cell Biochem. 112:1475-85; Choolani et al., 2003, Mol Hum Repro 9:227-35).

Cells with the desired morphology, nucleus to cell ratio, and fluorescent staining pattern(s) can be manually picked with the micromanipulator and placed in container (e.g., a 0.2 ml PCR tube) for downstream analysis purposes.

### 7. EXAMPLE 1

### Isolation and Analysis of fNRBCS

A 9 mL sample of maternal blood was spiked with 1 mL of cord blood and processed according to filtration protocol #1 and positive selection protocol #1. The filtration procedure was performed using a Pall Purecell® NEO filter and included three 50 mL 1× PBS chases and an elution with 50 mL of 1× PBS buffer. The MACS sorted cell population contained 2.0525×10⁸ total cells as measured with a Scepter™ cell counter.

The MACS sorted cell population was then split in half. One half was stained with an anti-CD235a-PE antibody, goat anti-mouse IgM Alexa 488 and DAPI, and the other half was stained with an anti-CD235a-PE antibody, goat anti-mouse IgM Alexa 488 and Cy5. The stained cell populations were each added to a glass bottomed petri dish. Each petri dish was then imaged using a fluorescence microscope and analyzed with NIS-Elements [symbol] (Nikon) to identify triple positive cells.

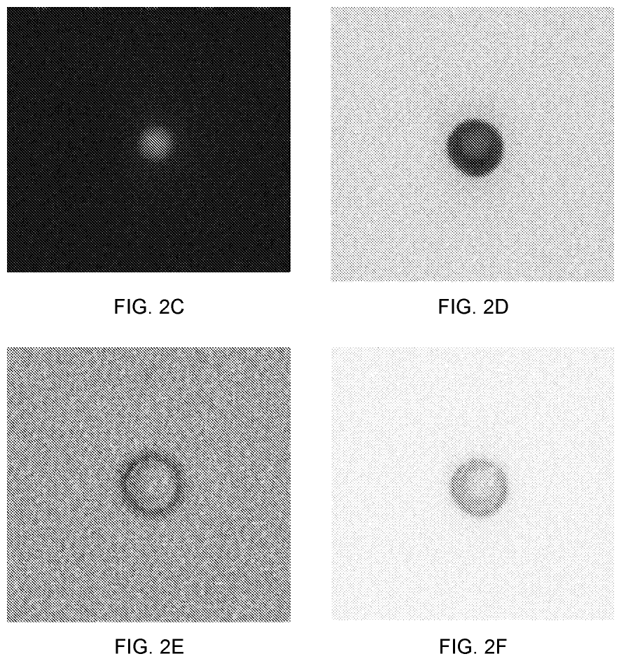

38 triple positive hits were identified from one area of the dish (a 4×4 grid of 16 total images) containing the cell population stained with Cy5. 34 of the 38 hits were determined to be nucleated red blood cells. 32 of the 34 nucleated red blood cells were intact nucleated red blood cells, while two had begun to disintegrate. Representative images of one of the nucleated red blood cells are shown in FIGS. 2A-2F.

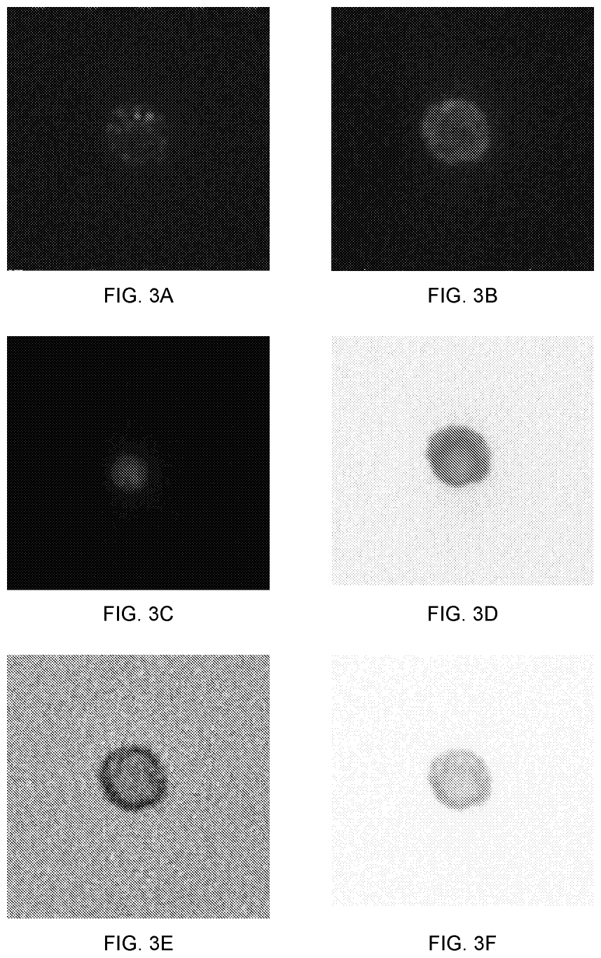

62 triple positive hits were identified from one area of the dish (a 4×4 grid of 16 total images) containing the cell population stained with DAPI. 55 of the 62 hits were determined to be nucleated red blood cells. 42 of the 55 nucleated red blood cells were intact nucleated red blood cells, while 13 had begun to disintegrate. Representative images of one of the nucleated red blood cell are shown in FIGS. 3A-3F.

### 8. SPECIFIC EMBODIMENTS

The present disclosure is exemplified by the specific embodiments below.

1. A method of enriching for fetal nucleated red blood cells (fNRBCs) from a biological sample, comprising:

(a) filtering the biological sample through a filter that retains fNRBCs on the filter and allows non-nucleated red blood cells if present in the sample to pass through the filter, to obtain a fNRBC-containing cell fraction;

(b) optionally, subjecting the fNRBC-containing cell fraction to magnetic activated cell sorting (MACS) using at least one fNRBC positive selection reagent to obtain a MACS-sorted cell population;

(c) fluorescently labeling cells in the fNRBC-containing cell fraction after step (a) or in the MACS-sorted cell population after step (b) with at least one fNRBC positive selection reagent to obtain a fluorescently labeled cell population; and

(d) performing micromanipulation on the fluorescently labeled cell population to isolate individual fNRBCs or groups of fNRBCs.

2. The method of embodiment 1, wherein the filter comprises a porous medium comprising a fibrous web.

3. The method of embodiment 2, wherein the fibrous web comprises a biocompatible polymer.

4. The method of any one of embodiments 1 to 3, wherein the filter i [symbol] reduction filter.

5. The method of any one of embodiments 1 to 4, wherein step (a) comprises applying the biological sample to the filter and collecting the fNRBC-containing fraction from the filter.

6. The method of embodiment 5, wherein the fNRBCs are collected by eluting the fNRBC-containing fraction from the filter with an elution buffer.

7. The method of embodiment 6, wherein the elution buffer is a buffer of physiological pH.

8. The method of embodiment 6 or embodiment 7, wherein the buffer is a saline buffer.

9. The method of any one of embodiments 6 to 8, wherein the elution buffer is a PBS buffer.

10. The method of any one of embodiments 5 to 9, further comprising one or more chases prior to collecting the fNRBC-containing fraction from the filter.

11. The method of embodiment 10, which comprises two chases.

12. The method of embodiment 10, which comprises three chases.

13. The method of any one of embodiments 1 to 12, which does not comprise step (b).

14. The method of any one of embodiments 1 to 12, which comprises step (b).

15. The method of embodiment 14, wherein step (b) utilizes at least one fNRBC positive selection reagent and step (c) utilizes at least two fNRBC positive selection reagents.

16. The method of embodiment 14, wherein step (b) utilizes at least one fNRBC positive selection reagent and step (c) utilizes at least three fNRBC positive selection reagents.

17. The method of any one of embodiments 14 to 16, wherein at least one fNRBC positive selection reagent of step (b) comprises monoclonal antibody 4B9.

18. The method of any one of embodiments 14 to 17, wherein at leas [symbol] positive selection reagent of step (b) comprises an anti-CD235a antibody.

19. The method of any one of embodiments 1 to 18, wherein at least one fNRBC positive selection reagent of step (c) comprises monoclonal antibody 4B9.

20. The method of any one of embodiments 1 to 19, wherein at least one fNRBC positive selection reagent of step (c) comprises an anti-CD235a antibody.

21. The method of any one of embodiments 1 to 20, wherein at least one fNRBC positive selection reagent of step (c) comprises a nuclear stain.

22. The method of embodiment 21, wherein the nuclear stain is DC-Ruby, DAPI, Hoechst 33342 or Cy5.

23. The method of any one of embodiments 1 to 22, which comprises, between steps (c) and (d), applying the fluorescently labeled cell population to a substrate.

24. The method of embodiment 23, wherein the substrate is suitable for fluorescence imaging.

25. The method of embodiment 23 or embodiment 24, wherein the substrate comprises polystyrene.

26. The method of embodiment 25, wherein the substrate is a single-well plate.

27. The method of any one of embodiment 23 or embodiment 24, wherein the substrate comprises glass.

28. The method of embodiment 27, wherein the substrate is a petri dish.

29. The method of any one of embodiments 1 to 28, further comprising performing fluorescence imaging on the fluorescently labeled cell population prior to step (d).

30. The method of embodiment 29, wherein the fluorescence imaging is performed using a fluorescence microscope.

31. The method of embodiment 30, wherein the fluorescence microscope is automated.

32. The method of any one of embodiments 1 to 31, wherein step (d) [symbol] performing micromanipulation to isolate individual fNRBCs or groups of fNRBCs labeled with all of the fNRBC positive selection reagents utilized in step (c).

33. The method of any one of embodiments 1 to 31, wherein the fNRBC positive selection reagents utilized in the method comprise monoclonal antibody 4B9, an anti-CD235a antibody, and a nuclear stain, and step (d) comprises performing micromanipulation to isolate individual fNRBCs or groups of fNRBCs labeled with monoclonal antibody 4B9, the anti-CD235a antibody, and the nuclear stain.

34. The method of any one of embodiments 1 to 33, which does not comprise a negative selection step.

35. The method of any one of embodiments 1 to 34, which does not comprise a fluorescence activated cell sorting (FACS) step.

36. The method of any one of embodiments 1 to 35, which does not comprise a density separation step.

37. The method of any one of embodiments 1 to 36, wherein the biological sample is maternal blood.

38. The method of any one of embodiments 1 to 36, wherein the biological sample is maternal blood diluted with a buffer.

39. The method of embodiment 38, further comprising diluting the maternal blood with the buffer.

40. The method of embodiment 38 or embodiment 39, wherein the buffer used to dilute the maternal blood is a PBS buffer.

41. The method of any one of embodiments 37 to 40, wherein the maternal blood is drawn between about four weeks and about thirty-eight weeks of gestation.

42. The method of embodiment 41, wherein the maternal blood is drawn between about six weeks and about twenty weeks of gestation.

43. The method of any one of embodiment 1 to 42, which further com [symbol] validating the identity of at least one fNRBC as a fetal cell.

44. A fNRBC obtained or obtainable by the method of any one of embodiments 1 to 43.

45. The fNRBC of embodiment 44, which is not fixed.

46. A cell population enriched in fNRBCs obtained or obtainable by the method of any one of embodiment 1 to 43.

47. The cell population enriched for fNRBCs of embodiment 46, which contains (a) at least 2, at least 5 or at least 10 and/or (b) up to 15, up to 25, up to 35, up to 50, or up to 75 fNRBCs enriched from maternal blood.

48. The cell population of embodiment 46 or embodiment 47, which is not fixed.

49. A method of detecting a fetal abnormality, comprising analyzing the fNRBC of embodiment 44 or embodiment 45 or at least one fNRBC from the cell population of any one of embodiments 46 to 48 for a fetal abnormality.

50. The method of embodiment 49, which further comprises enriching for fNRBCs according to the method of any one of embodiments 1 to 43 prior to analyzing.

51. The method of embodiment 49 or embodiment 50, which comprises analyzing a single fNRBC for the fetal abnormality.

52. The method of embodiment 49 or embodiment 50, which comprises analyzing a group of fNRBCs for the fetal abnormality.

53. The method of any one of embodiments 49 to 52, wherein the analyzing comprises a diagnostic assay.

54. The method of embodiment 53, wherein the diagnostic assay is a nucleic acid assay.

55. The method of embodiment 54, wherein the nucleic acid assay is a DNA assay, optionally where the DNA assay is carried out on a microarray.

56. The method of embodiment 54 or embodiment 55, wherein the n [symbol] assay is a FISH, PCR or DNA sequencing assay.

57. The method of embodiment 54, wherein the nucleic acid assay is an RNA assay, optionally where the RNA assay is carried out on a microarray.

58. The method of embodiment 57, wherein the RNA assay is an RT-PCR assay or a FISH assay.

59. The method of embodiment 53, wherein the diagnostic assay is a protein detection assay, optionally where the protein detection assay is carried out on a microarray.

60. The method of embodiment 59, wherein the protein is detected using an antibody.

61. The method of embodiment 53, wherein the diagnostic assay is a histological assay.

62. The method of any one of embodiments 53 to 61, which further comprises lysing or permeabilizing the fNRBC prior to carrying out the diagnostic assay.

63. The method of embodiment 51 or embodiment 52 which comprises performing whole genome amplification prior to analyzing.

64. The method of embodiment 51 or embodiment 52, which comprises amplifying a subset of the genome prior to analyzing.

65. The method of any one of embodiments 49 to 64, wherein the analysis comprises quantitative PCR.

66. The method of any one of embodiments 63 to 65, wherein the analysis is performed on a microarray.

67. The method of embodiment 51 or embodiment 52, which does not comprise PCR amplification.

68. The method of embodiment 67, which comprises rolling circle replication.

69. The method of embodiment 68, wherein the rolling circle replicati [symbol] least one fluorescent label.

70. The method of embodiment 67, wherein the analysis comprises fluorescence in-situ hybridization (FISH).

71. The method of embodiment 70, wherein the FISH utilizes at least one fluorescent probe.

72. The method of embodiment 51 or embodiment 52, which comprises analyzing one or more nucleic acid sequences from the single fNRBC or group of fNRBCs.

73. The method of embodiment 72, wherein analyzing the one or more nucleic acid sequences is performed by FISH, PCR or DNA sequencing.

74. The method of embodiment 72, wherein analyzing the one or more nucleic acid sequences is performed by quantitative PCR, real-time PCR or reverse transcriptase PCR.

75. The method of embodiment 72, wherein analyzing the one or more nucleic acids is performed by array comparative genomic hybridization (CGH).

76. The method of embodiment 72, wherein analyzing the one or more nucleic acid sequencese is performed by multiple annealing and looping based amplification cycles (MALBAC).

77. The method of embodiment 72, wherein analyzing the one or more nucleic acids includes a Next-Generation Sequencing technique or ultra-deep sequencing.

78. The method of embodiment 72, wherein the one or more nucleic acids are analyzed in a DNA microarray.

79. The method of embodiment 72, wherein the one or more nucleic acids are analyzed in a prenatal chromosomal microarray.

80. The method of embodiment 72, wherein the fetal abnormality is detected by a restriction fragment length polymorphism (RFLP).

81. The method of embodiment 72, wherein the fetal abnormality incl [symbol] microdeletion or a microduplication.

82. The method of embodiment 72, wherein the fNRBC comprises a telomere of increased or decreased length compared with a normal range of telomere lengths.

83. The method of embodiment 72, wherein analyzing the one or more nucleic acids comprises sequencing a stretch of a nucleic acid of a fNRBC.

84. The method of embodiment 83, wherein the stretch of the nucleic acid of the fNRBC is sequenced by next-generation sequencing technology or massively parallel sequencing

85. The method of any one of embodiments 49 to 84, which further comprises validating the fNRBC or fNRBCs as fetal cells.

86. The method of embodiment 85, wherein validation comprises performing short tandem repeat (STR) analysis, genetic fingerprinting or single nucleotide polymorphism (SNP) analysis.

87. The method of embodiment 85 or embodiment 86, wherein validation comprises comparing fNRBC DNA to maternal DNA.

88. The method of embodiment 85 or embodiment 86, wherein validation comprises comparing fNRBC DNA to both maternal and paternal DNA.

89. The method of any one of embodiments 49 to 88, wherein the fetal abnormality includes a single gene abnormality.

90. The method of any one of embodiments 49 to 89, wherein the fetal abnormality includes a single nucleotide polymorphism (SNP)

91. The method of any one of embodiments 49 to 88, wherein the fetal abnormality is trisomy 13, trisomy 18, trisomy 21, Down syndrome, neuropathy with liability to pressure palsies, neurofibromatosis, Alagille syndrome, achondroplasia, Huntington's disease, alpha-mannosidosis, beta-mannosidosis, metachromatic leucodystrophy, von Recklinghausen's disease, tuberous sclerosis complex, myotonic dystrophy, cystic fibrosis, sickle cell disease, tay-sachs disease, beta-thalassemia, mucopolysaccharidoses, phenylketonuria, citrullinuria, galactosemia, galactokinase and galactose 4-epimerase deficiency, adenine ph [symbol] transferase deficiency, methylmalonic acidurias, proprionic acidemia, Farber's disease, fucosidosis, gangliosidoses, gaucher's disease, I cell disease, mucolipidosis III, Niemann-Pick disease, sialidosis, Wolman's disease, Zellweger syndrome, cystinosis, factor X deficiency, ataxia telangiectasia, Bloom's syndrome, Robert's syndrome, xeroderma pigmentosum, fragile (X) syndrome, sex chromosome aneuploidy, Klinefelter's Syndrome, Turner's syndrome, XXX syndrome, steroid sulfatase deficiency, microphthalmia with linear skin defects, Pelizaeus-Merzbacher disease, testis-determining factor on Y, ornithine carbamoyl transferase deficiency, glucose 6-phosphate dehydrogenase deficiency, Lesch-Nyhan syndrome, Anderson-Fabry disease, hemophilia A, hemophilia B, Duchenne type muscular dystrophy, Becker type muscular dystrophy, dup(17)(p11.2p11.2) syndrome, 16p11.2 deletion, 16p11.2 duplication, Mitochondrial defect, dup(22)(q11.2q11.2) syndrome, Cat eye syndrome, Cri-du-chat syndrome, Wolf-Hirschhorn syndrome, Williams-Beuren syndrome, Charcot-Marie-Tooth disease, chromosome rearrangements, chromosome deletions, Smith-Magenis syndrome, Velocardiofacial syndrome, DiGeorge syndrome, 1p36 deletion, Prader-Willi syndrome, Azospermia (factor a), Azospermia (factor b), Azospermia (factor c), spina bifida, anencephaly, neural tube defect, microcephaly, hydrocephaly, renal agenesis, Kallmann syndrome, Adrenal hypoplasia, Angelman syndrome, cystic kidney, cystic hygroma, fetal hydrops, exomphalos and gastroschisis, diaphragmatic hernia, duodenal atresia, skeletal dysplasia, cleft lip, cleft palate, argininosuccinicaciduria, Krabbe's disease, homocystinuria, maple syrup urine disease, 3-methylcrotonyl coenzyme A, carboxylase deficiency, Glycogenoses, adrenal hyperplasia, hypophosphatasia, placental steroid sulphatase deficiency, severe combined immunodeficiency syndrome, T-cell immunodeficiency, Ehlers-Danlos syndrome, osteogenesis imperfect, adult polycystic kidney disease, Fanconi's anemia, epidermolysis bullosa syndromes, hypohidrotic ectodermal dysplasia, congenital nephrosis (Finnish type) and multiple endocrine neoplasia.

92. A method of enriching for fetal nucleated red blood cells (fNRBCs) from a biological sample, comprising:

(a) filtering the biological sample through a filter that retains fNRBCs on the filter and allows non-nucleated red blood cells if present in the sample to pass through the filter, to obtain a fNRBC-containing cell fraction;

(b) optionally, subjecting the fNRBC-containing cell fraction to magnetic activated cell sorting (MACS) using at least one fNRBC positive selection reagent to obtain a MACS-sorted cell population;

(c) fluorescently labeling cells in the fNRBC-containing cell fraction after step (a) or in the MACS-sorted cell population after step (b) with at least one fNRBC positive selection reagent to obtain a fluorescently labeled cell population; and

(d) performing micromanipulation on the fluorescently labeled [symbol] to isolate individual fNRBCs or groups of fNRBCs.

93. The method of embodiment 92, wherein the filter comprises a porous medium comprising a fibrous web.

94. The method of embodiment 93, wherein the fibrous web comprises a biocompatible polymer.

95. The method of any one of embodiments 92 to 94, wherein the filter is a leukocyte reduction filter.

96. The method of any one of embodiments 92 to 95, wherein step (a) comprises applying the biological sample to the filter and collecting the fNRBC-containing fraction from the filter.

97. The method of embodiment 96, wherein the fNRBCs are collected by eluting the fNRBC-containing fraction from the filter with an elution buffer.

98. The method of embodiment 97, wherein the elution buffer is a buffer of physiological pH.

99. The method of embodiment 97 or embodiment 98, wherein the buffer is a saline buffer.

100. The method of any one of embodiments 97 to 99, wherein the elution buffer is a PBS buffer.

101. The method of any one of embodiments 96 to 100, further comprising one or more chases prior to collecting the fNRBC-containing fraction from the filter.

102. The method of embodiment 101, which comprises two chases.

103. The method of embodiment 101, which comprises three chases.

104. The method of any one of embodiments 92 to 103, which does not comprise step (b).

105. The method of any one of embodiments 92 to 103, which comprises step (b).

106. The method of embodiment 105, wherein step (b) utilizes at least [symbol] positive selection reagent and step (c) utilizes at least two fNRBC positive selection reagents.

107. The method of embodiment 105, wherein step (b) utilizes at least one fNRBC positive selection reagent and step (c) utilizes at least three fNRBC positive selection reagents.

108. The method of any one of embodiments 105 to 107, wherein at least one fNRBC positive selection reagent of step (b) comprises monoclonal antibody 4B9.

109. The method of any one of embodiments 105 to 108, wherein at least one fNRBC positive selection reagent of step (b) comprises an anti-CD235a antibody.

110. The method of any one of embodiments 92 to 109, wherein at least one fNRBC positive selection reagent of step (c) comprises monoclonal antibody 4B9.

111. The method of any one of embodiments 92 to 110, wherein at least one fNRBC positive selection reagent of step (c) comprises an anti-CD235a antibody.

112. The method of any one of embodiments 92 to 111, wherein at least one fNRBC positive selection reagent of step (c) comprises a nuclear stain.

113. The method of embodiment 112, wherein the nuclear stain is DC-Ruby, DAPI, Hoechst 33342 or Cy5.

114. The method of any one of embodiments 92 to 113, which comprises, between steps (c) and (d), applying the fluorescently labeled cell population to a substrate.

115. The method of embodiment 114, wherein the substrate is suitable for fluorescence imaging.

116. The method of embodiment 114 or embodiment 115, wherein the substrate comprises polystyrene.

117. The method of embodiment 116, wherein the substrate is a single-well plate.

118. The method of any one of embodiment 114 or embodiment 115, wherein the substrate comprises glass.

119. The method of embodiment 118, wherein the substrate is a petri dish.

120. The method of any one of embodiments 92 to 119, further compri [symbol] fluorescence imaging on the fluorescently labeled cell population prior to step (d).

121. The method of embodiment 120, wherein the fluorescence imaging is performed using a fluorescence microscope.

122. The method of embodiment 121, wherein the fluorescence microscope is automated.

123. The method of any one of embodiments 92 to 122, wherein step (d) comprises performing micromanipulation to isolate individual fNRBCs or groups of fNRBCs labeled with all of the fNRBC positive selection reagents utilized in step (c).

124. The method of any one of embodiments 92 to 122, wherein the fNRBC positive selection reagents utilized in the method comprise monoclonal antibody 4B9, an anti-CD235a antibody, and a nuclear stain, and step (d) comprises performing micromanipulation to isolate individual fNRBCs or groups of fNRBCs labeled with monoclonal antibody 4B9, the anti-CD235a antibody, and the nuclear stain.

125. The method of any one of embodiments 92 to 124, which does not comprise a negative selection step.

126. The method of any one of embodiments 92 to 125, which does not comprise a fluorescence activated cell sorting (FACS) step.

127. The method of any one of embodiments 92 to 126, which does not comprise a density separation step.

128. The method of any one of embodiments 92 to 127, wherein the biological sample is maternal blood.

129. The method of any one of embodiments 92 to 127, wherein the biological sample is maternal blood diluted with a buffer.

130. The method of embodiment 129, further comprising diluting the maternal blood with the buffer.

131. The method of embodiment 129 or embodiment 130, wherein the [symbol] dilute the maternal blood is a PBS buffer.

132. The method of any one of embodiments 128 to 131, wherein the maternal blood is drawn between about four weeks and about thirty-eight weeks of gestation.

133. The method of embodiment 132, wherein the maternal blood is drawn between about six weeks and about twenty weeks of gestation.

134. The method of any one of embodiment 1 to 133, which further comprises validating the identity of at least one fNRBC as a fetal cell.

135. A fNRBC obtained or obtainable by the method of any one of embodiments 92 to 134.

136. The fNRBC of embodiment 135, which is not fixed.

137. A cell population enriched in fNRBCs obtained or obtainable by the method of any one of embodiment 1 to 134.

138. The cell population enriched for fNRBCs of embodiment 137, which contains (a) at least 2, at least 5 or at least 10 and/or (b) up to 15, up to 25, up to 35, up to 50, or up to 75 fNRBCs enriched from maternal blood.

139. The cell population of embodiment 137 or embodiment 138, which is not fixed.

140. A method of detecting a fetal abnormality, comprising analyzing the fNRBC of embodiment 135 or embodiment 136 or at least one fNRBC from the cell population of any one of embodiments 46 to 48 for a fetal abnormality.

141. The method of embodiment 140, which further comprises enriching for fNRBCs according to the method of any one of embodiments 92 to 134 prior to analyzing.

142. The method of embodiment 140 or embodiment 141, which comprises analyzing a single fNRBC for the fetal abnormality.

143. The method of embodiment 140 or embodiment 141, which comprises analyzing a group of fNRBCs for the fetal abnormality.

144. The method of embodiment 142 or embodiment 143 which comp [symbol] whole genome amplification prior to analyzing.

145. The method of embodiment 142 or embodiment 143, which comprises amplifying a subset of the genome prior to analyzing.

146. The method of any one of embodiments 140 to 145, wherein the analysis comprises quantitative PCR.

147. The method of embodiment 142 or embodiment 143, which does not comprise PCR amplification.

148. The method of embodiment 147, which comprises rolling circle replication.

149. The method of embodiment 148, wherein the rolling circle replication utilizes at least one fluorescent label.

150. The method of any one of embodiments 140 to 149, wherein the analysis is performed on a microarray.

151. The method of embodiment 147, wherein the analysis comprises fluorescence in-situ hybridization (FISH).

152. The method of embodiment 151, wherein the FISH utilizes at least one fluorescent probe.

153. The method of any one of embodiments 140 to 152, which further comprises validating the fNRBC or fNRBCs as fetal cells.

154. The method of embodiment 153, wherein validation comprises performing short tandem repeat (STR) analysis, genetic fingerprinting or single nucleotide polymorphism (SNP) analysis.

155. The method of embodiment 153 or embodiment 154, wherein validation comprises comparing fNRBC DNA to maternal DNA.

156. The method of embodiment 153 or embodiment 154, wherein validation comprises comparing fNRBC DNA to both maternal and paternal DNA.

While various specific embodiments have been illustrated and described, it will be appreciated that various changes can be made without departing from the spirit and scope of the disclosure(s).

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A method of enriching for fetal nucleated red blood cells (fNRBCs) from a biological sample, comprising:
(a) filtering the biological sample through a filter that retains fNRBCs on the filter and allows non-nucleated red blood cells if present in the sample to pass through the filter, to obtain a fNRBC-containing cell fraction;
(b) optionally, subjecting the fNRBC-containing cell fraction to magnetic activated cell sorting (MACS) using at least one fNRBC positive selection reagent to obtain a MACS-sorted cell population;
(c) fluorescently labeling cells in the fNRBC-containing cell fraction after step (a) or in the MACS-sorted cell population after step (b) with at least one fNRBC positive selection reagent to obtain a fluorescently labeled cell population; and
(d) performing micromanipulation on the fluorescently labeled cell population to isolate individual fNRBCs or groups of fNRBCs.

2. The method of claim 1, wherein the filter comprises a porous medium comprising a fibrous web.

3. The method of claim 2, wherein the fibrous web comprises a biocompatible polymer.

4. The method of any one of claims 1 to 3, wherein the filter is a leukocyte reduction filter.

5. The method of any one of claims 1 to 4, wherein step (a) comprises applying the biological sample to the filter and collecting the fNRBC-containing fraction from the filter.

6. The method of claim 5, wherein the fNRBCs are collected by eluting the fNRBC-containing fraction from the filter with an elution buffer.

7. The method of claim 6, wherein the elution buffer is a buffer of physiological pH.

8. The method of claim 6 or claim 7, wherein the buffer is a saline buffer.

9. The method of any one of claims 6 to 8, wherein the elution buffer is a PBS buffer.

10. The method of any one of claims 5 to 9, further comprising one o [symbol] prior to collecting the fNRBC-containing fraction from the filter.

11. The method of claim 10, which comprises two or three chases.

12. The method of any one of claims 1 to 11, which does not comprise step (b).

13. The method of any one of claims 1 to 11, which comprises step (b).

14. The method of claim 13, wherein step (b) utilizes at least one fNRBC positive selection reagent and step (c) utilizes at least two fNRBC positive selection reagents.

15. The method of claim 13, wherein step (b) utilizes at least one fNRBC positive selection reagent and step (c) utilizes at least three fNRBC positive selection reagents.

16. The method of any one of claims 13 to 15, wherein at least one fNRBC positive selection reagent of step (b) comprises monoclonal antibody 4B9.

17. The method of any one of claims 13 to 16, wherein at least one fNRBC positive selection reagent of step (b) comprises an anti-CD235a antibody.

18. The method of any one of claims 1 to 17, wherein at least one fNRBC positive selection reagent of step (c) comprises monoclonal antibody 4B9.

19. The method of any one of claims 1 to 18, wherein at least one fNRBC positive selection reagent of step (c) comprises an anti-CD235a antibody.

20. The method of any one of claims 1 to 19, wherein at least one fNRBC positive selection reagent of step (c) comprises a nuclear stain, which is optionally DC-Ruby, DAPI, Hoechst 33342 or Cy5.

21. The method of any one of claims 1 to 20, which comprises, between steps (c) and (d), applying the fluorescently labeled cell population to a substrate.

22. The method of claim 21, wherein the substrate is suitable for fluorescence imaging.

23. The method of claim 21 or claim 22, wherein the substrate comprises polystyrene, optionally wherein the substrate is a single-well plate.

24. The method of any one of claim 21 or claim 22, wherein the subs [symbol] glass, optionally wherein the substrate is a petri dish.

25. The method of any one of claims 1 to 24, further comprising performing fluorescence imaging on the fluorescently labeled cell population prior to step (d).

26. The method of claim 25, wherein the fluorescence imaging is performed using a fluorescence microscope, optionally wherein the fluorescence microscope is automated.

27. The method of any one of claims 1 to 26, wherein step (d) comprises performing micromanipulation to isolate individual fNRBCs or groups of fNRBCs labeled with all of the fNRBC positive selection reagents utilized in step (c).

28. The method of any one of claims 1 to 26, wherein the fNRBC positive selection reagents utilized in the method comprise monoclonal antibody 4B9, an anti-CD235a antibody, and a nuclear stain, and step (d) comprises performing micromanipulation to isolate individual fNRBCs or groups of fNRBCs labeled with monoclonal antibody 4B9, the anti-CD235a antibody, and the nuclear stain.

29. The method of any one of claims 1 to 28, which does not comprise a negative selection step.

30. The method of any one of claims 1 to 29, which does not comprise a fluorescence activated cell sorting (FACS) step.

31. The method of any one of claims 1 to 30, which does not comprise a density separation step.

32. The method of any one of claims 1 to 31, wherein the biological sample is maternal blood or maternal blood diluted with a buffer, optionally wherein the buffer is a PBS buffer.

33. The method of claim 32, wherein the maternal blood is drawn between about four weeks and about thirty-eight weeks of gestation.

34. The method of claim 33, wherein the maternal blood is drawn between about six weeks and about twenty weeks of gestation.

35. The method of any one of claims 1 to 34, which further comprises [symbol] identity of at least one fNRBC as a fetal cell.

36. A fNRBC obtained or obtainable by the method of any one of claims 1 to 35, which is optionally not fixed.

37. A cell population enriched in fNRBCs obtained or obtainable by the method of any one of claims 1 to 35, optionally which contains (a) at least 2, at least 5 or at least 10 and/or (b) up to 15, up to 25, up to 35, up to 50, or up to 75 fNRBCs enriched from maternal blood, and/or optionally which is not fixed.

38. A method of detecting a fetal abnormality, comprising analyzing the fNRBC of claim 36 or at least one fNRBC from the cell population of claim 37 for a fetal abnormality.

39. The method of claim 38, which further comprises enriching for fNRBCs according to the method of any one of claims 1 to 35 prior to analyzing.

40. The method of claim 38 or claim 39, which comprises analyzing a single fNRBC for the fetal abnormality.

41. The method of claim 38 or claim 39, which comprises analyzing a group of fNRBCs for the fetal abnormality.

42. The method of claim 40 or claim 41 which comprises performing whole genome amplification prior to analyzing.

43. The method of claim 40 or claim 41, which comprises amplifying a subset of the genome prior to analyzing.

44. The method of any one of claims 38 to 43, wherein the analysis comprises quantitative PCR.

45. The method of claim 40 or claim 41, which does not comprise PCR amplification and optionally comprises:
(a) rolling circle replication, optionally wherein the rolling circle replication utilizes at least one fluorescent label; or
(b) fluorescence in-situ hybridization (FISH), optionally utilizing at least one fluorescent probe.

46. The method of any one of claims 38 to 45, wherein the analysis i [symbol] a microarray.

47. The method of any one of claims 38 to 46, which further comprises validating the fNRBC or fNRBCs as fetal cells.

48. The method of claim 47, wherein validation comprises performing short tandem repeat (STR) analysis, genetic fingerprinting or single nucleotide polymorphism (SNP) analysis.

49. The method of claim 47 or claim 48, wherein validation comprises comparing fNRBC DNA to maternal DNA or comparing fNRBC DNA to both maternal and paternal DNA.
